(19) United States
(12) Patent Application Publication
(10) Pub. No.: US 2024/0153616 A1
(43) Pub. Date: May 9, 2024

(54) SYSTEMS AND METHODS FOR DEEP LEARNING MODEL ANNOTATION USING SPECIALIZED IMAGING MODALITIES

(71) Applicant: PathAI, Inc., Boston, MA (US)

(72) Inventors: Justin Lee, Cambridge, MA (US); Seyed Mohammad Mirzadeh, Somerville, MA (US); Tan Huu Nguyen, Hopkinton, MA (US); Waleed Tahir, Dorchester, MA (US); Jun Zhang, Sudbury, MA (US); Yibo Zhang, Brookline, MA (US)

(73) Assignee: PathAI, Inc., Boston, MA (US)

(21) Appl. No.: 18/494,157

(22) Filed: Oct. 25, 2023

Related U.S. Application Data
(60) Provisional application No. 63/489,013, filed on Mar. 8, 2023.
(60) Provisional application No. 63/425,981, filed on Nov. 16, 2022.
(60) Provisional application No. 63/422,410, filed on Nov. 3, 2022.

(51) Int. Cl. G16H 30/40; G06T 5/50; G06T 7/00; G06T 7/30; G06V 20/70; G16H 50/20

(52) U.S. Cl. CPC G16H 30/40; G06T 5/50; G06T 7/0012; G06T 7/30; G06V 20/70; G16H 50/20; G06T 2207/20081; G06T 2207/30024

(57) ABSTRACT

In some aspects, a method, a system, or a non-transitory computer-readable storage medium are described for using a machine learning (ML) model to obtain annotations of a pathology slide image obtained in a first imaging modality, where the ML model is trained based in part on images obtained from a second imaging modality different from the first imaging modality. The first imaging modality is a conventional scanner for whole-slide images (WSI). The second imaging modality may include one or more of multispectral imaging (MSI), polarization imaging, quantitative phase imaging, or a combination thereof. The trained ML model can generate annotations that include more details with higher accuracy in comparison to annotating based on the WSI images alone.

### CROSS-REFERENCE TO RELATED APPLICATIONS

This application claims the benefit of U.S. Provisional Application Ser. No. 63/425,981, filed on Nov. 16, 2022, under Attorney Docket No. P1112.70019US00, entitled “STROMAL SUBDIVISIONAL MODEL,” U.S. Provisional Application Ser. No. 63/422,410, filed on Nov. 3, 2022, under Attorney Docket No. P1112.7002011500, entitled “SYSTEMS AND METHODS FOR DEEP LEARNING MODEL ANNOTATION USING SPECIALIZED IMAGING MODALITIES,” and U.S. Provisional Application Ser. No. 63/489,013, filed on Mar. 8, 2023, under Attorney Docket No. P1112.7002111500, entitled “SYSTEMS AND METHODS FOR QUANTITATIVE POLARIZATION IMAGING,” each of which is hereby incorporated herein by reference in its entirety.

### BACKGROUND

Deep learning techniques may be used to process pathology images (e.g., whole-slide images) to identify tissues and cells that may be associated with certain types of diseases. Training datasets for training a deep learning model typically include manual annotations of whole-slide images with the aid of pathologists. For example, a training dataset may include a plurality of annotated whole-slide images each including one or more annotations that are manually annotated by one or more pathologists, where an annotation may associate certain cell-type, tissue-type or other physical properties or structure in the whole-slide images with portions of the whole-slide images.

A manual annotation process can be expensive, biased, and time-consuming (e.g., intractable to get exhaustive annotations of a substance, e.g., collagen across an entire whole-slide image). For example, there can be low inter-rater expert agreement and low intra-rater agreement over multiple reads of slides with respect to portions in the images that lack substance human perceptible details or stain variability. Inaccurate annotations resulting from a manual annotation process will cause the performance of the deep learning techniques to decrease.

### SUMMARY

Some embodiments relate to a method comprising: using a machine learning (ML) model to obtain annotations of a pathology slide image obtained in a first imaging modality; wherein the ML model is trained based in part on images obtained from a second imaging modality different from the first imaging modality.

In some embodiments, the first imaging modality is configured to image a slide based on light source of visible wavelengths and absorption of light by tissue.

In some embodiments, the second imaging modality comprises one or more of multispectral imaging (MSI), polarization imaging, quantitative phase imaging, or a combination thereof.

In some embodiments, the method further comprises training the ML model, using a plurality of pairs of first image and second images; wherein the first image in the pair is obtained from the first modality imaging of a first pathology slide; and the second image in the pair is generated based on a second modality imaging of a second pathology slide corresponding to the first pathology slide.

In some embodiments, the second pathology slide and the first pathology slide are a same physical slide.

In some embodiments, the training further includes registering the first image and the second image in each of the pairs of first image and second image.

In some embodiments, the registering includes aligning the first image and the second image in each of the pairs.

In some embodiments, the second image in the pair is an annotation image comprising a plurality of objects each associated with a respective portion of the second image.

In some embodiments, the method further comprises generating the annotation image by processing an image captured by the second modality imaging over a physical slide.

In some embodiments, the method further comprises generating the annotation image based on a plurality of images captured by the second modality imaging over a physical slide.

In some embodiments, the method further comprises generating HIFs from the annotations.

In some embodiments, the method further comprises using a second ML to predict cell/tissue from the pathology slide image; and generating the HIFs based additionally on the predicted cell/tissue.

In some embodiments, the method further comprises predicting a disease based on the HIFs, using a statistical model.

In some embodiments, the annotations of the pathology slide image comprise heatmaps or labels of tissues/cells in the pathology slide image.

Some embodiments relate to method comprising using a machine learning (ML) model to obtain annotations of a pathology slide image of a first type; wherein the ML model is trained based in part on training pathology slide images of a second type different from the first type.

In some embodiments, the first type of image is obtained from a stained slide; and the second type of image is a stain-invariant image obtained from a triplex slide.

In some embodiments, the second type of image is a phase image.

Some embodiments relate to method for denoising images of samples, comprising performing multi-spectral polarization imaging of a sample to generate a polarization image of the sample; segmenting the polarization image to form a plurality of image segments; obtaining spectral characteristics associated with at least some of the plurality of image segments, wherein obtaining the spectral characteristics comprises performing spectral analysis on the at least some of the plurality of image segments; and identifying, using the respective spectral characteristics, a first subset of the at least some of the plurality of image segments as including a substance of interest and a second subset of the at least some of the plurality of image segments as including artifacts.

In some embodiments, the substance of interest comprises collagen.

In some embodiments, the substance of interest comprises an amyloid.

In some embodiments, the artifact comprises calcium.

In some embodiments, the artifact comprises metal.

In some embodiments, performing multi-spectral polarization imaging of the sample comprises illuminating the sample with a plurality of light emitting diodes (LEDs) emitting light at mutually distinct wavelength simultaneously.

In some embodiments, performing multi-spectral polarization imaging of the sample comprises illuminating the sample with a plurality of light emitting diodes (LEDs) emitting light at mutually distinct wavelength sequentially.

In some embodiments, segmenting the polarization image to form the plurality of image segments comprises segmenting the polarization image pixel-wise so that each image segment corresponds to a pixel of the polarization image.

In some embodiments, segmenting the polarization image to form the plurality of image segments comprises segmenting the polarization image pixel-wise so each image segment corresponds to a group of pixels of the polarization image.

In some embodiments, the method further comprises generating a denoised image of the sample using the first subset.

In some embodiments, the method further comprises providing the denoised image of the sample as input to a machine learning model.

In some embodiments, performing spectral analysis on the at least some of the plurality of image segments comprises obtaining spectra associated with the at least some of the plurality of image segments and comparing the spectra to known spectra associated with a plurality of known samples.

Some embodiments relate to system for denoising images of samples, comprising a multi-spectral polarization imaging apparatus configured to generate a polarization image of a sample; and a computer hardware processor configured to segment the polarization image to form a plurality of image segments; obtain spectral characteristics associated with at least some of the plurality of image segments, wherein obtaining the spectral characteristics comprises performing spectral analysis on the at least some of the plurality of image segments; and identify, using the respective spectral characteristics, a first subset of the at least some of the plurality of image segments as including a substance of interest and a second subset of the at least some of the plurality of image segments as including artifacts.

In some embodiments, the substance of interest comprises collagen.

In some embodiments, the substance of interest comprises an amyloid.

In some embodiments, the artifact comprises calcium.

In some embodiments, the artifact comprises metal.

In some embodiments, the multi-spectral polarization imaging apparatus comprises a plurality of light emitting diodes (LEDs) emitting light at mutually distinct wavelength simultaneously, and wherein the system further comprises a controller configured to cause the LEDs to emit light simultaneously.

In some embodiments, the multi-spectral polarization imaging apparatus comprises a broadband light source, and a plurality of narrowband color filters.

In some embodiments, the multi-spectral polarization imaging apparatus comprises a plurality of light emitting diodes (LEDs) emitting light at mutually distinct wavelength simultaneously, and wherein the system further comprises a controller configured to cause the LEDs to emit light in accordance with time-domain multiplexing (TDM).

In some embodiments, segmenting the polarization image to form the plurality of image segments comprises segmenting the polarization image pixel-wise so that each image segment corresponds to a pixel of the polarization image.

In some embodiments, segmenting the polarization image to form the plurality of image segments comprises segmenting the polarization image pixel-wise so each image segment corresponds to a group of pixels of the polarization image.

In some embodiments, the processor is further configured to generate a denoised image of the sample using the first subset.

In some embodiments, performing spectral analysis on the at least some of the plurality of image segments comprises obtaining spectra associated with the at least some of the plurality of image segments and comparing the spectra to known spectra associated with a plurality of known samples.

Some embodiments relate to method comprising using a machine learning (ML) model to segment a pathology slide image into a plurality of portions, wherein the ML model is configured to divide an image region into a plurality of regions corresponding to a plurality of stromal sub-types comprising at least densely inflamed stroma, densely fibroblastic stroma, mature stroma, immature stroma, and elastosis; and each of the plurality of segmented portions corresponds to one of the plurality of stromal sub-types.

In some embodiments, the ML model is a first ML model, and wherein the method further comprises using a second ML model to determine one or more cancer-associated stroma areas in the pathology slide image; and providing the one or more cancer-associated stroma areas as input to the first ML model.

In some embodiments, the method further comprises determining one or more human interpretable features (HIFs) based at least in part on the plurality of segmented regions; and predicting prognosis, gene expression, and/or other clinically relevant features based at least in part on the one or more HIFs.

In some embodiments, the prognosis, gene expression, and/or other clinically relevant features each is associated with one or more of: NSCLC, pancreatic adenocarcinoma, cholangiocarcinoma, colorectal carcinoma, urothelial carcinoma, and breast cancer.

In some embodiments, the one or more HIFs include one or more of: total area of a stromal sub-type, area proportion of a stromal sub-type over total tissue, area proportion of a stromal sub-type over total stroma, area proportion of a stromal sub-type over cancer, ratio of total area of a stromal sub-type to another stromal sub-type, and/or total area or area proportion of a combination of two or more stromal sub-types over total tissue, total stroma or cancer.

In some embodiments, the ML model is a first ML model, and wherein the method further comprises: using a second ML model to predict one or more cells in the pathology slide image; and determining the one or more human interpretable features (HIFs) based additionally on the one or more predicted cells.

In some embodiments, the one or more HIFs additionally include cellular HIFs comprising one or more of: total count of a cell type in a stromal sub-type, count proportion of a cell type over another cell type in a stromal sub-type, or density of a cell type in a stromal sub-type, and a combination of total count/count proportion/density of cell type(s) in two or more stromal sub-types.

In some embodiments, the plurality of stromal sub-types comprise one or more additional sub-types.

In some embodiments, the pathology slide image is a H&E-stained image.

Some embodiments relate to method comprising using a first ML model to determine one or more cancer-associated stroma areas in a pathology slide image; using a second ML model to segment the pathology slide image into a plurality of portions based at least in part on the one or more cancer-associated stroma areas as input to the second ML model, wherein: the second ML model is configured to divide an image region into a plurality of regions corresponding to a plurality of stromal sub-types; and each of the plurality of segmented portions corresponds to one of the plurality of stromal sub-types; using a third ML model to predict one or more cells in the pathology slide image; and predicting prognosis, gene expression, and/or other clinically relevant features associated with a solid tumor disease based on the plurality of segmented portions and the predicted one or more cells in the pathology slide image.

In some embodiments, the plurality of stromal sub-types comprise at least densely inflamed stroma, densely fibroblastic stroma, mature stroma, immature stroma, and elastosis.

In some embodiments, predicting the prognosis, gene expression, and/or other clinically relevant features associated with the solid tumor disease based at least in part on the plurality of segmented portions in the pathology slide image comprises determining one or more human interpretable features (HIFs) based at least in part on the plurality of segmented regions; and predicting the prognosis, gene expression, and/or other clinically relevant features based at least in part on the one or more HIFs.

In some embodiments, the solid tumor disease comprises one or more of: NSCLC, pancreatic adenocarcinoma, cholangiocarcinoma, colorectal carcinoma, urothelial carcinoma, and breast cancer.

In some embodiments, the one or more HIFs include one or more of: total area of a stromal sub-type, area proportion of a stromal sub-type over total tissue, area proportion of a stromal sub-type over total stroma, area proportion of a stromal sub-type over cancer, ratio of total area of a stromal sub-type to another stromal sub-type, and/or total area or area proportion of a combination of two or more stromal sub-types over total tissue, total stroma or cancer.

In some embodiments, the one or more HIFs additionally include cellular HIFs comprising one or more of: total count of a cell type in a stromal sub-type, count proportion of a cell type over another cell type in a stromal sub-type, or density of a cell type in a stromal sub-type, and a combination of total count/count proportion/density of cell type(s) in two or more stromal sub-types.

In some embodiments, the pathology slide image is a H&E-stained image.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Various aspects of at least one embodiment are discussed below with reference to the accompanying figures, which are not intended to be drawn to scale. Where technical features in the figures, detailed description or any claim are followed by reference signs, the reference signs have been included for the sole purpose of increasing the intelligibility of the figures, detailed description, and claims. Accordingly, neither the reference signs nor their absence is intended to have any limiting effect on the scope of any claim elements. For purposes of clarity, not every component may be labeled in every figure. The figures are provided for the purposes of illustration and explanation and are not intended as a definition of the limits of the systems and methods described herein. In the figures:

FIG. 1-1A shows components of a training system for training a machine learning model to generate annotations using various modalities of imaging, in accordance with some embodiments of the technology described herein.

FIG. 1-1B shows components of a deploying system for prognostic prediction, diagnostic prediction, or patient outcome prediction of certain diseases using one or more statistical models, in accordance with some embodiments of the technology described herein.

FIGS. 1-2A shows illustrative components of the training and deployment systems of FIG. 1-1A, in accordance with some embodiments of the technology described herein.

FIGS. 1-2B shows illustrative components of the training and deployment systems of FIG. 1-1B, in accordance with some embodiments of the technology described herein.

FIG. 1-3 shows application of the models described herein to prediction of Non-Alcoholic Steatohepatitis (NASH) fibrosis stage, in accordance with some embodiments of the technology described herein.

FIG. 1-4 shows imaging fibrosis in NASH tissues, in accordance with some embodiments of the technology described herein.

FIG. 2-1 shows components of a deploying system for prognostic prediction, diagnostic prediction, or patient outcome prediction of solid tumor using at least a stroma subdivision model, in accordance with some embodiments of the technology described herein.

FIG. 2-2 shows examples of stromal sub-types showing heterogeneity of cancer stroma, in accordance with some embodiments of the technology described herein.

FIG. 2-3A shows an example of PDAC tissues, in accordance with some embodiments of the technology described herein.

FIG. 2-3B shows an example of PDAC stromal sub-types, in accordance with some embodiments of the technology described herein.

FIG. 2-4 illustrates an example of stroma subdivisional model performance in LUAD and LUSC, in accordance with some embodiments of the technology described herein.

FIG. 3-1 illustrates a system for multispectral polarization microscopy, in accordance with some embodiments of the technology described herein.

FIG. 3-2 illustrates an image of a sample obtained using polarization microscopy, in accordance with some embodiments of the technology described herein.

FIG. 3-3A is a block diagram illustrating a method for denoising images, in accordance with some embodiments of the technology described herein.

FIG. 3-3B illustrates spectra associated with multiple image segments of an image obtained using a polarization imaging apparatus, in accordance with some embodiments of the technology described herein.

FIG. 3-4 illustrates an image of a sample obtained using polarization microscopy and upon application of denoising, in accordance with some embodiments of the technology described herein.

### DETAILED DESCRIPTION

### I. Annotation Generation

Modern computer vision methods present the potential for rapid, reproducible, and cost-effective clinical and molecular predictions. Over the past decade, the quantity and resolution of digitized histology slides has dramatically improved. At the same time, the field of computer vision has made significant strides in pathology image analysis, including automated prediction of Gleason scoring in prostate cancer, and mutational subtypes in non-small cell lung cancer. In addition to achieving diagnostic sensitivity and specificity metrics that match or exceed those of human pathologists, automated computational pathology can also scale to service resource-constrained settings where few pathologists are available. As a result, there may be opportunities to integrate these technologies into the clinical workflows of developing countries.

However, end-to-end deep learning models that infer outputs directly from raw images present significant risks for clinical settings, including fragility of machine learning models to dataset shift, adversarial attack, and systematic biases present in training data. Many of these risks stem from the well-known problem of poor model interpretability. “Black-box” model predictions are difficult for users to interrogate and understand, leading to user distrust. Without reliable means for understanding when and how vulnerabilities may become failures, computational methods may face difficulty achieving widespread adoption in clinical settings.

To address issues with the conventional approaches, the inventors have developed solutions for automated computation of human-interpretable image features (HIFs) to predict disease severity. The described HIF-based prediction models may minor the pathology workflow of searching for distinctive, stage-defining features under a microscope and offer opportunities for pathologists to validate intermediate steps and identify failure points. In addition, the described HIF-based solutions may enable incorporation of histological knowledge and expert pixel-level annotations which increases predictive power. Studied HIFs span a wide range of visual features, including cell and nucleus morphologies, shapes and sizes of tissue regions of normal colon as well as pathological tissue regions characteristic of inflammatory bowel disease, tissue textures and the spatial distributions of inflammatory cells, liver disease, cancer fibrosis, chromogenic mIHC, or other diseases.

The inventors have appreciated and acknowledged that deep machine learning models (e.g., cell-type model and/or tissue-type model) may be used to predict cells/tissues in a slide image. These predicted cells/tissues may be subsequently used to determine HIFs for the HIF-based solutions. Most deep learning techniques rely on manual annotation processes, which rely on pathologist experts to identify physical properties, e.g., texture, pattern, color (e.g., in stained slides), and associate them with corresponding cells/tissues or other structures in the slide images. As such, variations may exist in human annotated images because the perceptions of pathologist experts over certain features and properties in the slide images may be subjective. Additionally, it can be difficult for pathologists to distinguish signals from conventional imaging techniques (e.g., identifying/annotating collagen in an H&E image is extremely difficult). Further, what pathologists can distinguish in slide images can be limited by stain variability (e.g., trichrome is over/under-stained) or the level of substance detail needed for accurate annotation (e.g., annotating perisinusoidal fibrosis). For example, in the context of chromogenic multiplex IHC, pathologists (who all see color differently) may have difficulty identifying marker co-expression, especially in the presence of strong chromogen spectral overlap.

Accordingly, the inventors have developed methods and systems for training and deploying an annotation machine learning model using specialized imaging modalities that are employed to better elucidate desired signal (e.g., SHG for collagen and multispectral imaging for stain separation in chromogenic multiplex IHC) that can provide the level of substance detail as needed for better annotations.

In a first aspect of the present disclosure, a method, system or a non-transitory computer readable storage medium is provided that is configured to use a machine learning (ML) model to obtain annotations of a pathology slide image obtained in a first imaging modality; wherein the ML model is trained based in part on images obtained from a second imaging modality different from the first imaging modality. For example, the first imaging modality may be a conventional whole-slide imaging (WSI) scanner. An example of the conventional WSI scanner is a Leica Aperio AT2 scanner. The second imaging modality may include non-conventional imaging, such as polarization imaging, multispectral imaging (MSI), quantitative phase imaging, and/or a combination of these various imaging modalities. An example of a combined imaging modality may include quantitative multimodal anisotropy imaging (QMAI) which combines polarization imaging with MSI and quantitative phase imaging. The second imaging modality may generate an image from a physical slide to reveal structured substances that may not be visible in images obtained from the first imaging modality. For example, QMAI may be used to highlight structured substances such as collagen in tissue in the slide images.

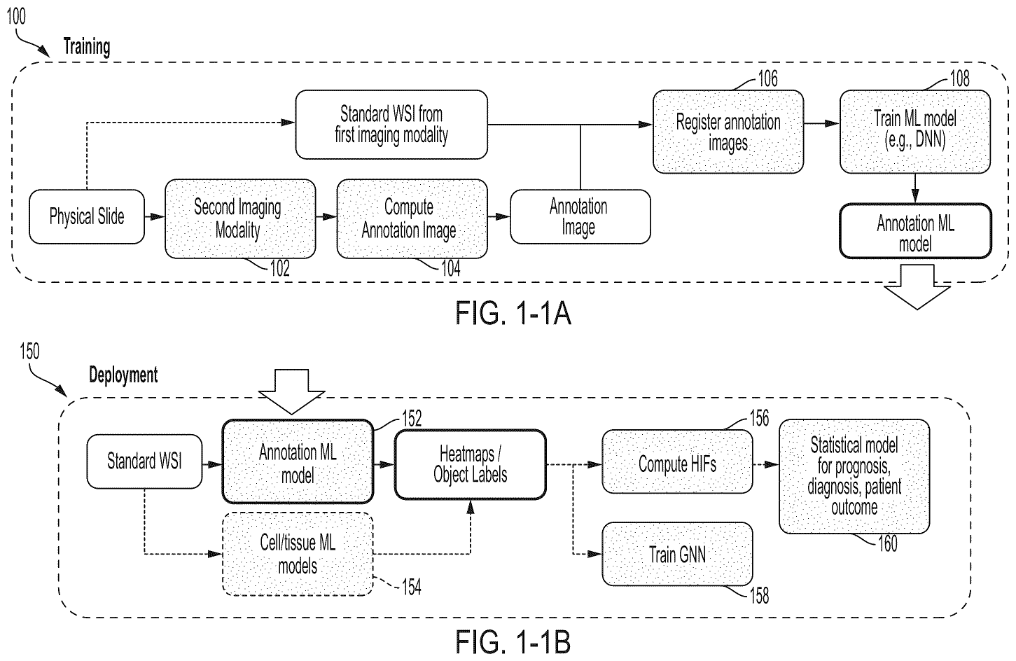

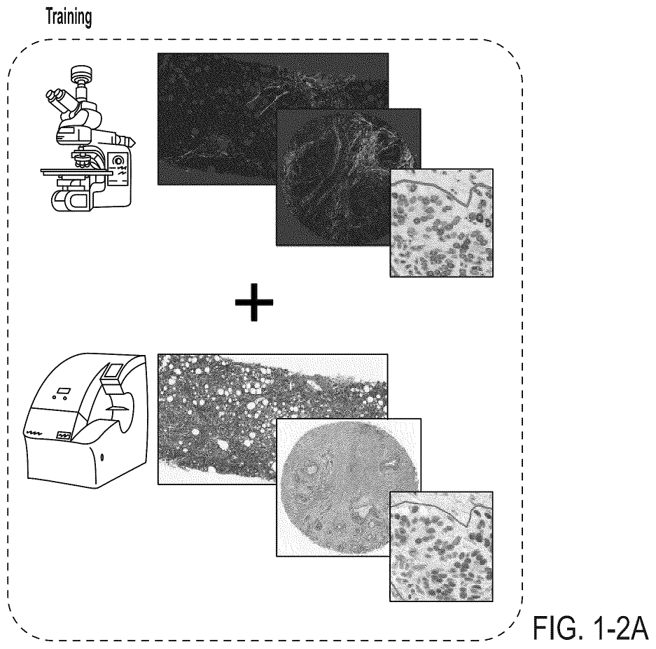

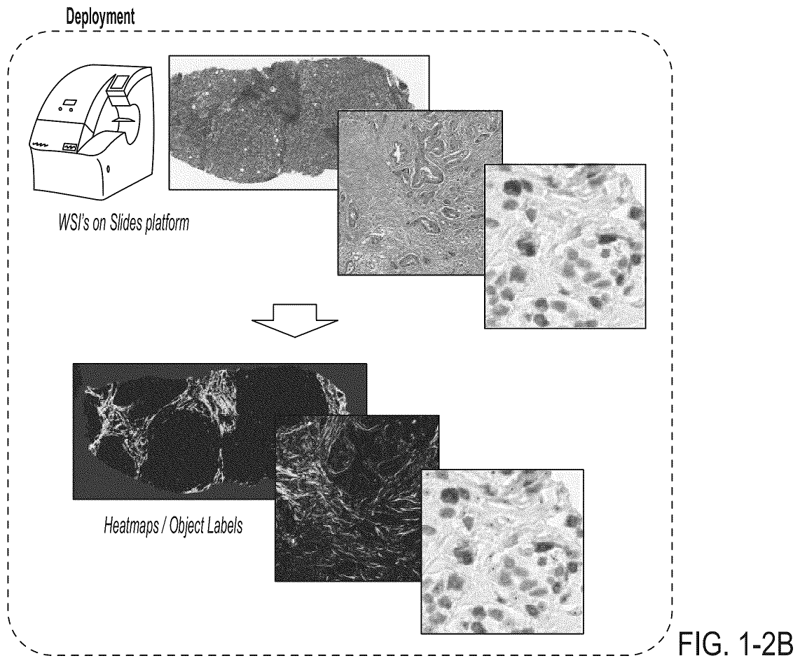

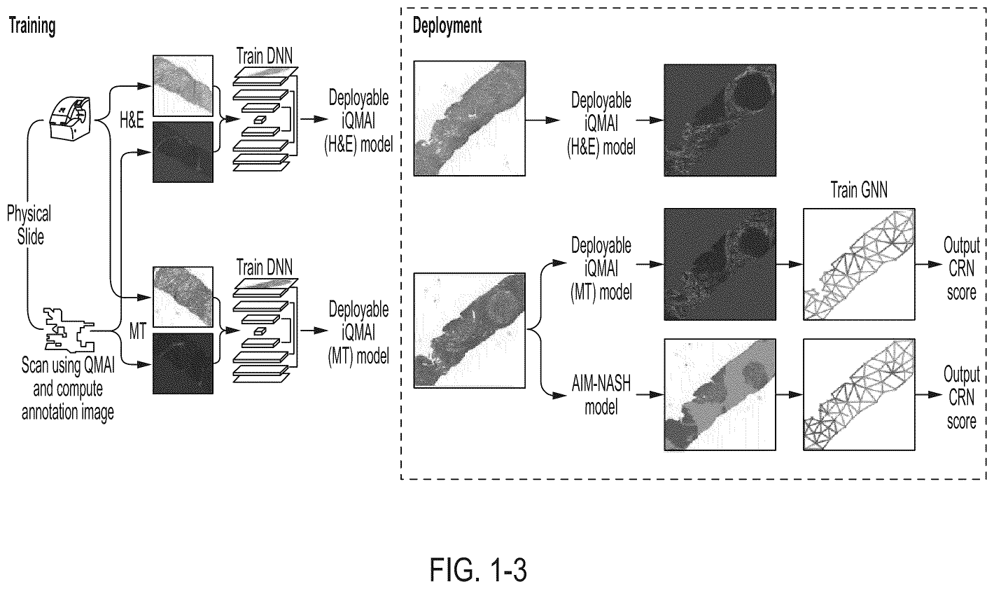

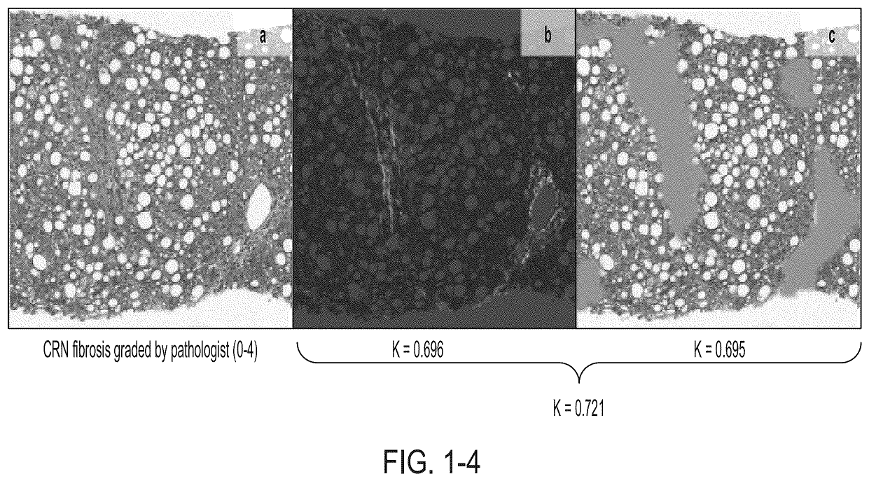

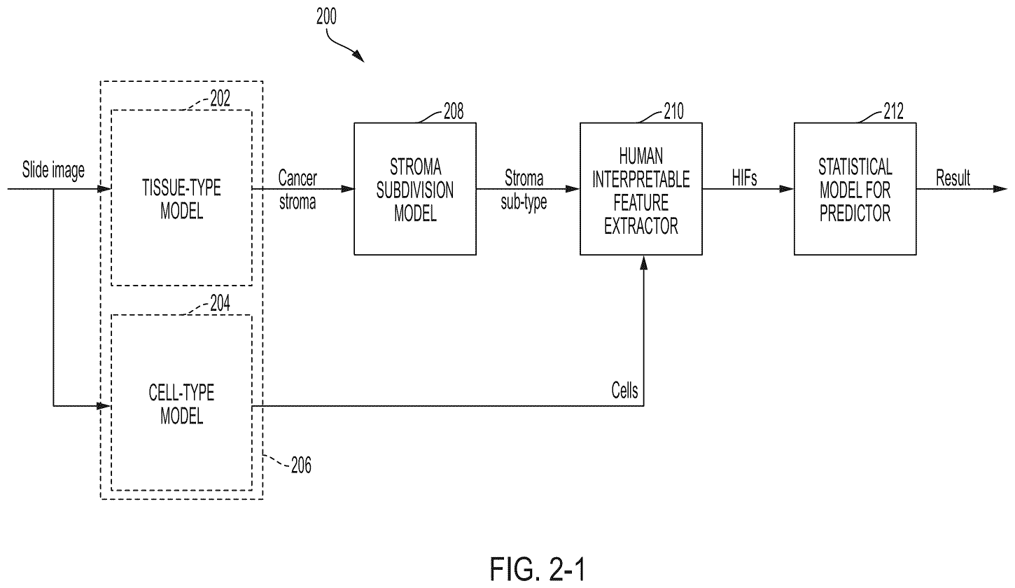

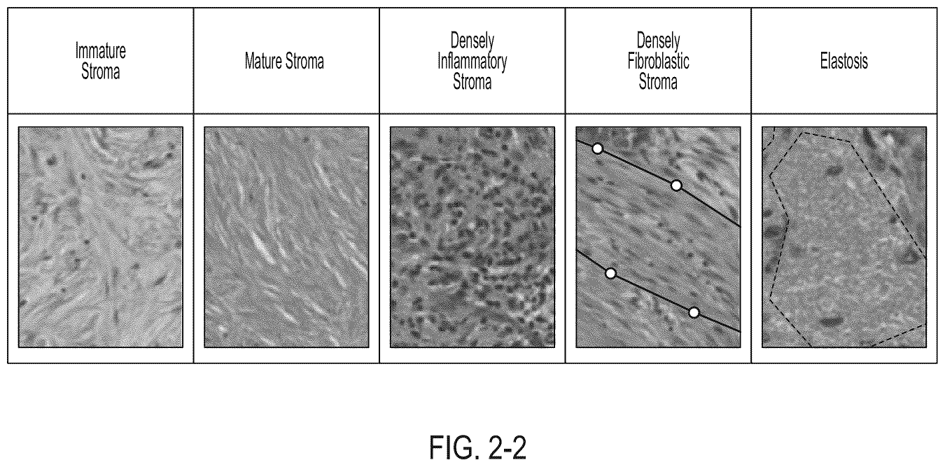

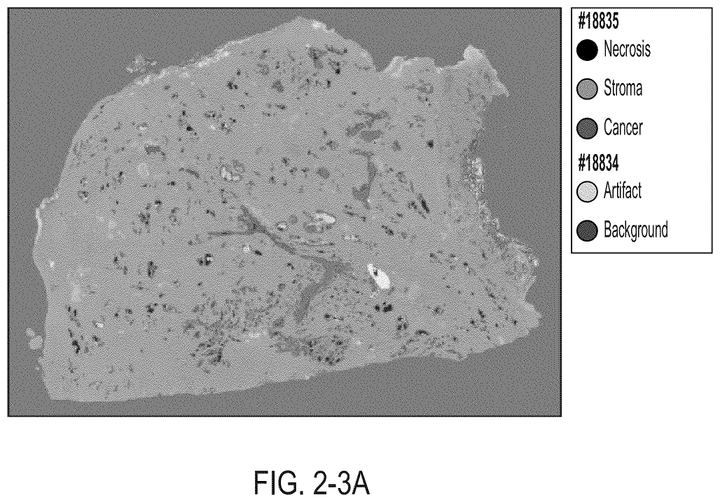

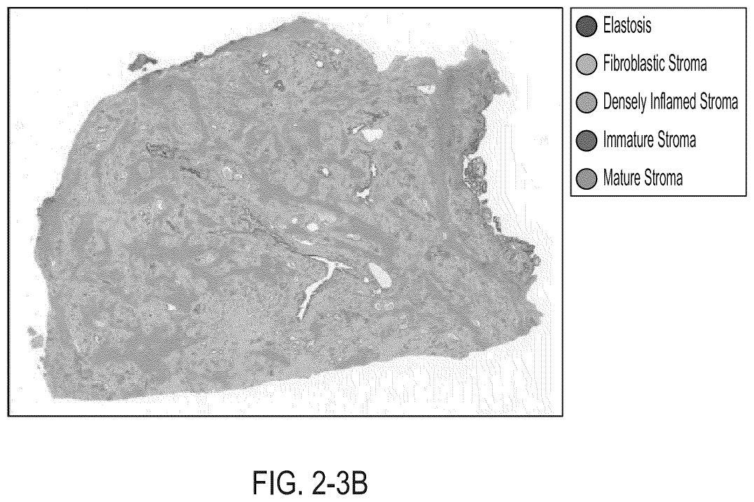

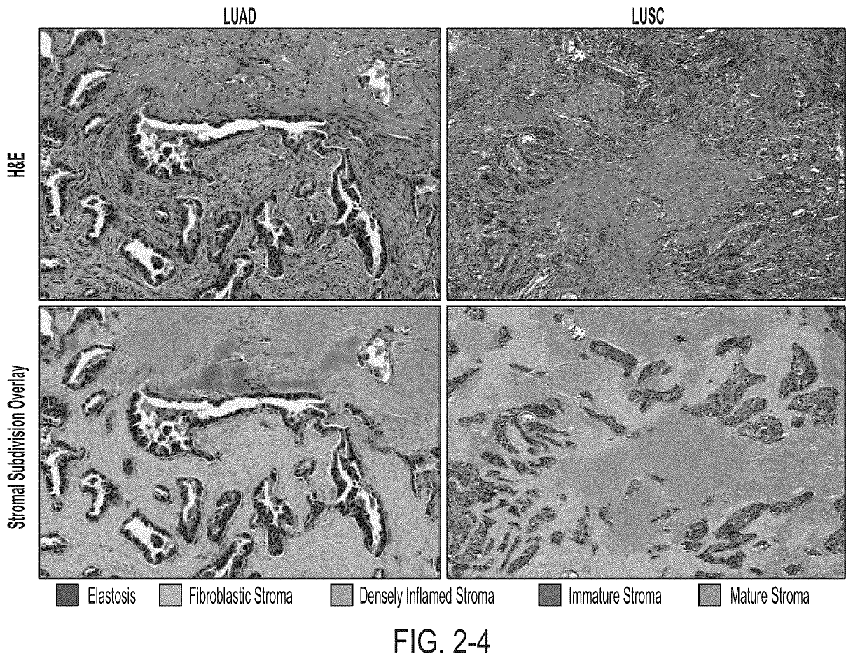

In some embodiments, the training dataset for training the annotation ML model may include a plurality of pairs of a first image and a second image, where the first image in the pair is obtained from the first modality imaging of a first pathology slide and the second image in the pair is generated based on the second modality imaging of a second pathology slide corresponding to the first pathology slide. For example, a physical slide is scanned using the first imaging modality to generate the first image. In some examples, the second pathology slide may be the first pathology slide. For example, the same physical slide scanned by the first imaging modality may be imaged using the second imaging modality to generate the second image. In some embodiments, the physical slide may be unstained or stained (e.g., H&E-stained, or MT-stained). Accordingly, the ML model can be trained using the plurality of pairs of the first and second images and configured to, for a given input image from the first imaging modality (e.g., a WSI image scanned from a pathology slide), generate an output image, which may have similar features as if the slide was imaged using the second imaging modality (without actually imaging the slide using the second imaging modality). Details of the training are further described in the present disclosure with reference to FIGS. 1-1A through 1-2B.

The ML model as described above and further herein in the present disclosure may be any suitable deep learning network, such as a deep neural network (e.g., a CNN having multiple layers). The training of the ML model may use any suitable optimization method, e.g., gradient descent or other suitable methods.

In some embodiments, before training the ML model, the first and second images in each pair of training images may be registered (e.g., aligned), such that the pixels of corresponding tissues/cells (or other patterns, physical properties) in the first and second images are aligned.

In some embodiments, the second image in the pair of first and second images may be generated by processing one or more raw image(s) captured in the second imaging modality over the physical slide. In a non-limiting example, the second image may be generated by processing an MSI image obtained from the MSI modality over the physical slide. The MSI image may be a multi-spectral image having eight channels, where the second image may include four colors (corresponding to the color contribution to the stain) or other suitable colors. The color (channel) conversion may be performed using any suitable color conversion methods, an example of which will be described further herein. In another non-limiting example, the second image may be generated by combining multiple images captured by the second imaging modality over a physical slide at two different angles. The second image in the pair of first and second images may be generated by combining two MSI images captured at two different angles within the field of view (FOV).

In some embodiments, the images obtained from the second imaging modality may each be an annotation image that includes a plurality of annotations each including an association between an object in the second image (structures/tissues) and a corresponding portion of the second image (e.g., a pixel, a group of pixels, a region in the image). In some embodiments, an annotation in the image may be manually annotated by a human expert (pathologist). For example, a pathologist may identify one or more objects (e.g., tissues) in the second image and label them. Alternatively, and/or additionally, an annotation in the image may be annotated automatically. For example, an image obtained from the second imaging modality may be processed in that the pixels may be classified into one of a tissue type (or other structure) by thresholding, where each of the classified tissue type (or other structure) is labeled. Alternatively, the annotations in the training images may be generated semi-automatically while allowing a human expert to correct/add/alter machine generated annotations.

Once the annotation ML model is trained, it can be stored and deployed for generating annotations.

In some embodiments, the method, system, or non-transitory computer readable storage medium as described herein above are further configured to use the inferred annotations from the trained ML model to generate a heatmap/object labels for further processing. For example, the results from the trained annotation ML model may include a heatmap showing cluster(s) of tissues/cells, and/or one or more regions associated with a corresponding tissue/cell. Additionally, the heatmap/object labels may be enhanced by using a second ML model configured to predict cell/tissue from the pathology slide image that was also provided as input to the annotation ML model as described herein.

In some embodiments, the method, system, or non-transitory computer readable medium as described herein above are further configured to determine human interpretable features (HIFs) based on the heatmaps/object labels. For example, HIFs may be determined based on the shapes, cell count, size ratio or other physical properties identified from the inferred annotations. The HIFs to be determined may depend on the disease the system is designed to predict. Examples of HIFs are further described in the present disclosure.

In some embodiments, the method, system, or non-transitory computer readable storage medium as described herein above are further configured to perform prognostic prediction, diagnostic prediction, and/or patient outcome prediction using a statistical model. The statistical model may be a suitable type of non-linear regression model such as a random forest regression model, a support vector regression model, or an adaptive basis function regression model.

In some embodiments, the method, system, or non-transitory computer readable storage medium as described herein above are further configured to use a third ML (e.g., a graph neural network, GNN) which can be trained from heatmaps/object labels to infer prognostic prediction, diagnostic prediction, and/or patient outcome prediction of certain diseases. Thus, the heatmaps/object labels obtained from the annotation ML model (and/or in combination with the output of the cell/tissue ML) may be provided as input to the third ML, which infers prognostic prediction, diagnostic prediction, or patient outcome prediction of certain diseases.

In a second aspect of the disclosure, a method, system, and non-transitory computer storage medium is provided that is configured to use a machine learning (ML) model to obtain annotations of a pathology slide image of a first type, where the ML model is trained based in part on training pathology slide images of a second type different from the first type. For example, the first type of image may be a WSI image obtained from a stained slide, whereas the second type of image may be a phase image of the WSI image. The training dataset for training the annotation ML model may include a plurality of pairs of first image and second image, where the first image is the phase image (e.g., of a triplex slide) and the second image is a heatmap image. The heatmap images in the training dataset may also be annotated manually, automatically, or semi-automatically with the combination of annotation by human experts.

Once the pairs of the first and second images are generated, they can be used to train the annotation ML model in a similar as described with respect to the first aspect of the disclosure. For example, the first image and the second image may be registered (aligned) and together used to train the annotation ML model. Similarly, once trained in the manner described above, the annotation ML model may be deployed in a similar manner as described in the first aspect.

Still other aspects, embodiments, and advantages of these exemplary aspects and embodiments, are discussed in detail below. The accompanying drawings are included to provide illustration and a further understanding of the various aspects and embodiments and are incorporated in and constitute a part of this specification. The drawings, together with the remainder of the specification, serve to explain principles and operations of the described and claimed aspects and embodiments.

FIG. 1-1A shows aspects of a training system 100 for training a machine learning model to generate annotations using various modalities of imaging, in accordance with some embodiments of the technology described herein. As shown, the training system 100 may include a second imaging modality 102 as previously described. The second imaging modality may include a non-conventional imaging modality such as polarization imaging, multispectral imaging, quantitative phase imaging, or a combination thereof. Thus, in training system 100, a first image may be obtained from a physical slide (e.g., a cover-slipped slide of fixed pathology specimens) using a conventional imaging technique. A conventional imaging technique may include imaging based on light source of visible wavelengths and absorption of light by tissue. For example, a WSI scanner (e.g., a Leica Aperio AT2 scanner) can be used to obtain the first image from the first imaging modality. The second image may be obtained from the second imaging modality that is configured to image on the same physical slide. The second imaging modality may include one or more other imaging modalities, such as, for example, MSI, polarization imaging, phase imaging, or a combination thereof. Each of these imaging modalities can operate standalone, or in combination with other imaging modalities, and may be configured to operate in various ways to suit different applications.

In some examples, an implementation of polarization imaging may include two cross-polarizers that are rotated (while maintaining relative orientation, e.g., 90 degrees) to probe structural anisotropy of the tissue. Images at two or more such angles may be combined to make a quantitative polarization image. Other polarization-based techniques may also be possible. An example of polarization imaging that may be used as part of the second imaging modality is described below in connection with FIGS. 3-1 through 3-4.

In some examples, an implementation of multispectral imaging may use a multi-wavelength light source and bandpass filters (e.g., LED's at various wavelengths across the spectrum, each filtered by 5-10 nm filters). In some scenarios, when a light source has sufficient power (or if a longer scan time is permissible), an implementation may include a single white light source and bandpass filters (with longer exposure time). In some examples, the longer exposure time may be possible because the tissues in the physical slides are static.

In some examples, an implementation of quantitative phase imaging may be based on “transport of intensity,” where an image stack is acquired with the illumination aperture stopped down to make the light source partially coherent. A mathematical problem, such as the “transport of intensity equation” can be solved/inverted to recover the quantitative phase of the system.

The second imaging modality may generate an image from a physical slide to reveal structured substances that may not be visible in images obtained from the first imaging modality. For example, as described above, QMAI may be used to combine polarization imaging with MSI and quantitative phase imaging, to produce combined image(s) that highlight structured substances such as collagen in tissue in the slide images.

As described, in the various imaging modalities, including the conventional WSI scanning, the cover-slipped slides of fixed pathology specimens may be used. Additionally, and/or alternatively, certain imaging modalities may also accommodate various slide types. For example, phase imaging and polarization imaging may be used to image living specimens that are unstained. MSI may be used to image a stained slide, although multispectral polarization and multispectral phase imaging may not require a stained slide. In some examples, polarization imaging may be best used for slides that do not have plastic cover slipped as noisy signals may be picked up from cover-slip scratches and the polymer tape itself.

With further reference to FIG. 1-1A, training system 100 may include a component 104 for further processing the second image(s) obtained from the second imaging modality to generate an annotation image to be used for training. It is appreciated the various methods may be used to compute the annotation images, depending on the imaging modality and application. For example, the computing may include removing noise in an image obtained from any of the imaging modality. In another example, the second imaging modality may be polarization imaging, where the annotation image may be a heatmap annotation image (showing clustering of certain tissues). The annotation image may be generated by combining (e.g., adding) a plurality of polarization images obtained at different angles. For example, an annotation image may be obtained by adding two polarization images that are obtained at 0 degree and 45 degrees, respectively.

In some examples, the second imaging modality may be MSI, where the annotation image may include a collection of point annotations. In a non-limiting example, a raw image captured via MSI may include eight channels (representing colors), which may be converted to a 4-channel representation of different stain contributions per pixel via stain separation/color deconvolution. An eight-to-four matrix may be determined using control slides or estimated from experimental slides, and later used for converting an MSI image to an annotation image, where the annotation image may have fewer channels than the MSI raw image. For example, for every stain, a color composition is known for each of the multispectral wavelengths in MSI. Thus, an eight-to-four matrix can be determined, with each column representing a different stain with each row corresponding to how absorptive/responsive that stain is at any of the probed eight wavelengths.

In some examples, the image obtained from MSI may be converted to a four-channel image, each channel representing a different stain contribution. In some embodiments, each image channel may be processed (individually, or in combination) to identify likely locations of objects (e.g., tissues) for each of these stains. For example, pixels in each channel image may be binarized using a threshold, to generate a binary image. Then, connected components may be determined from the binary image, where the connected components may represent an object. Then, a point label may be determined based on the centroid of the identified object. Whereas various image processing techniques are described, it is appreciated other pre-processing or post-processing techniques may be used. For example, each channel image (or binary image from thresholding) may be pre-processed for noise remove or outliers/spurious signal removal.

In some embodiments, the above described methods can be performed for other imaging modalities, or in conjunction with another ML model. For example, a ML model may be trained to detect/segment cell nuclei in each channel image, where the detected/segmented cell nuclei can be further analyzed for object labeling. In other embodiments, a ML model may be trained to provide point-wise labels of stain status (+ or −) for each object/nucleus based on pathologist annotations of the stain-separated images. Whereas various embodiments of image annotation described above can be automatic, it is appreciated that the annotation image computation component 104 may include fully automatic annotation (e.g., based on the various embodiments described herein), in combination with manual annotations (by human experts).

With further reference to FIG. 1-1A, training system 100 may optionally include a registration component 106 configured to register the first image (e.g., WSI from the first imaging modality) and the second image (e.g., annotation image generated based on the image obtained from the second imaging modality 102). Registration of the first and second images aligns the pixels of corresponding tissues/cells (or other patterns, physical properties) in the first and second images. In a non-limiting example, the first image and the second image are aligned using cross-correlation. Grid search may also be used for proper angular orientation and cross-correlation. In other variations, feature-based approach may be used to align two images, where a plurality of feature points are first identified (e.g., via an algorithm such as SIFT or ORB) followed by matching and fitting those putative matches to a model (e.g., via RANSAC) to determine how the images are misaligned (including displacement, possible distortion, etc.).

Although alignment between two images in a pair of images for the training is described, it is appreciated that registration (alignment) may be optional. For example, in QMAI modality which combines multiple imaging techniques, alignment between the various images obtained from multiple imaging modalities may not be needed because the same physical slide is being used.

With further reference to FIG. 1-1A, the plurality of pairs of first and second images are registered (at 106) and provided to the training component (108) for training the annotation ML model. The annotation ML model may be a deep neural network, and various training techniques for training a neural network can be used. It is appreciated that other ML models may be used.

FIG. 1-1B shows aspects of a deploying system for prognostic prediction, diagnostic prediction, or patient outcome prediction using one or more statistical models, in accordance with some embodiments of the technology described herein. Deployment system 150 may include an annotation ML model 152 for generating annotations from a standard WSI image. In some embodiments, the annotation ML model 152 may be trained in the training system 100 as described with reference to FIG. 1-1A. The annotation ML model 152 may be previously stored, for example, once it is trained. During deployment, the annotation ML model 152 is provided with standard WSI images (e.g., images captured from the first imaging modality previously described with reference to FIG. 1-1A) as input and executed to infer annotations, as output, such as heatmaps/object labels.

It is appreciated that the annotation ML model described herein does not compromise any existing or other components in a ML-based prediction system. For example, another ML model (e.g., cell/tissue model 154) may be provided in the deployment system 150, where the model 154 is configured to predict cells/tissues from WSI images. These predicted cells/tissues may be used to enhance the heatmaps/object labels produced by the annotation ML model 152. For example, additional cells/tissues inferred from the model 154 may be added/combined in the output from the annotation ML model.

With further reference to FIG. 1-1B, the deployment system 150 may further include a human-interpretable feature (HIF) component 156 configured to generate multiple HIFs, which may be used to generate a statistical model 160 for prognostic prediction, diagnostic prediction, and/or patent outcome prediction.

As shown in FIG. 1-1B, alternatively, and/or additionally, a neural network (e.g., graph neural network 158) may be trained from the heatmaps/object labels provided by the annotation ML model to be able to perform prognostic prediction, diagnostic prediction, and/or patent outcome prediction.

In some embodiments, a variation of the systems described in FIGS. 1-1A and 1-1B may include a system that is configured to use a machine learning (ML) model to obtain annotations of a pathology slide image of a first type, wherein the ML model is trained based in part on training pathology slide images of a second type different from the first type. In some examples, the first type of image is WSI image (such as what is described above and obtained from a conventional WSI scanner), and the second type of image may be a phase image such as a phase image of a triplex slide. An example of a triplex slide is a slide that's chromogenically stained with three different marker-specific stains (e.g., ER, PR, Ki67),

In training the annotation ML model, the first image in a pair of training images may be a phase image obtained from a triplex slide and the second image in the pair may be an annotation image, such as tissue heatmap image. Multiple pairs of training images may be obtained in a similar manner, and then used to train the annotation ML model. The trained annotation ML model is thus configured to generate a tissue heatmap for each given phase image (obtained from a respective triplex slide). In a non-limiting example, the annotation ML model may be trained by identifying epithelium/stroma in phase images of triplex slides (unstained no-counterstain slide). A phase image may be stain-invariant. For example, the phase images of slide images of various stains may be similar. Thus, during deployment, instead of providing the stained image as input to the annotation ML model, the system may provide the phase image of the slide as input to the annotation ML model. In such configuration, the annotation ML model can be used in a stain-agnostic way on phase images of other slides to infer annotations. The resulting annotations may include details that may be not available from a slide image of a particular stain (or certain stains) alone.

The variation of the system as described above may also operate in a similar manner as previously described with reference to FIGS. 1-1A and 1-1B. For example, once the pairs of training images including different types (e.g., stain-invariant images such as phase images) are generated, the images in the pairs can be annotated, registered and the annotation ML model can be trained in a similar manner as described in embodiments of FIG. 1-1A (see 104, 106, 108). Similarly, the trained annotation ML model using the variation of the training system may be used in the same deployment system as described herein (see 150 in FIG. 1-1B).

FIGS. 1-2A and 1-2B show illustrative components of the training and deployment systems of FIGS. 1-1A and 1-1B, respectively, in accordance with some embodiments of the technology described herein. FIG. 1-2A shows the first imaging modality (e.g., acquired WSI images from a WSI scanner, such as a Leica Aperio AT2 scanner) at the bottom, and the second imaging modality on the top. Each of the imaging modalities generate a first image and a second image in a plurality of image pairs. For example, FIG. 1-2A shows three pairs of training images, each pair including a WSI image from the first imaging modality (bottom) and a corresponding image from the second imaging modality (top). These pairs of training images are provided to train the annotation ML model such as what is described in FIG. 1-1A. For example, the first pair (left) includes a WSI image (bottom) and a QMAI image (top); the second pair (middle) includes a WSI image (bottom) and a QMAI image (top); and a third pair (right) includes a WSI image (bottom) and a MSI image (top).

FIG. 1-2B shows the deployment system and examples of annotated images using the trained annotation ML model. For example, the input (top) to the annotation ML model includes WSI images obtained from a conventional scanner. The output (bottom) of the annotation ML model includes the inferred annotation images.

As shown in in FIG. 1-2B, although no second imaging modality as described in training system 100 is required in the deployment system, the output of the annotation ML model will include tissues/objects as similarly seen in images from the second imaging modality (e.g., polarization images) used for training the annotation ML model. In other words, the annotation ML model learns the associations between features in the WSI images and features in the images from the second imaging modality and, for any new WSI image, can infer the features/objects/annotations as if the images from the second imaging modality were used.

The advantages of the annotation ML model described herein thus become apparent in that the annotations will include more details and will be more accurate as compared to annotating based on the WSI images alone. Additionally, once the annotation ML model is trained, no second imaging modality is required during deployment (prediction). As the second imaging modality may require a physical slide to be imaged, and the system may be expensive/bulky, the deployment of the annotation ML model, which does not require the second imaging modality, becomes much more feasible and usable. Further, the WSI images may be previously scanned and stored in the database for accessing, thus, the deployment of the annotation ML model may not require any physical side.

A wide range of applications may be implemented using the various embodiments described in the present disclosure, where the advantages of these embodiments will become more apparent. In a non-limiting application, the system may be used to obtain images in MSI imaging modality and use multispectral images to guide annotation of (or automatically annotate) chromogenic mIHC images. The results may be used as super-annotations of matched conventional (RGB) whole-slide images to predict marker status. In another non-limiting application, polarization images may be used in combination with pathologist annotation (e.g., region of interest—ROI annotation) of perisinusoidal fibrosis to get a detailed perisinusoidal fibrosis annotation.

The method and system described herein may be used as an assisting tool for manual annotation or automatic annotation. The various embodiments as described herein are advantageous over existing systems as the annotation ML model that is trained based in part on the second imaging modality enables detailed annotations, such as shown in FIG. 1-2B. This enables users to access highly specific data (e.g., fiber-level collagen heatmap, improved stain-separation in mIHC) for downstream analyses, all without the need for a second imaging modality or physical slide access.

Throughout this disclosure, a neural network is used as an exemplary basis for a statistical model (e.g., a deep learning model) that may be used in accordance with some embodiments. However, it should be appreciated that other types of statistical models may alternatively be used, and embodiments are not limited in this respect. Other types of statistical models that may be used include a support vector machine, a neural network, a regression model, a random forest, a clustering model, a Bayesian network, reinforcement learning, metric learning, a genetic algorithm, or another suitable statistical model.

Various computer architectures/systems may be used to implement the training system (100 in FIG. 1-1A) and deployment system (150 in FIG. 1-1B), and/or any components thereof.

As an example, the models described herein were used in the context of non-alcoholic steatohepatitis (NASH). In general, staging fibrosis severity in non-alcoholic steatohepatitis (NASH) requires pathologist review of tissue stained to visualize collagen. The accuracy of staging can be affected by both stain quality and variability of pathologists' interpretation of the stain. Quantitative Multimodal Anisotropy Imaging (QMAI) can highlight collagen in tissue and can be used in quantification and staging of NASH fibrosis. AIM-NASH is a machine learning model developed by the inventors based on the models described above using 26,000 pathologist annotations on whole slide images (WSI) of Masson's Trichrome (MT)-stained tissue to accurately and reproducibly predict NASH Clinical Research Network (CRN) fibrosis stage. Here, QMAI provides detailed, unbiased annotations of fibrosis on MT-stained tissue that are used to train deep neural network (DNN)-based ML models to infer a QMAI fibrosis pattern (iQMAI), which is then used by graph neural networks (GNNs) to predict slide level CRN fibrosis scores. ML models based on DNNs were trained to predict a QMAI-like fibrosis pattern in tissue using 14 slides of liver tissue from patients with NASH from a clinical laboratory. All sides were stained with MT and scanned on a Leica Aperio AT2 scanner and imaged by QMAI imaging to create paired digitized images of the same slide. DNNs were trained using the paired QMAI and AT2 scanned WSI, as shown in FIG. 1-3. FIG. 1-3 further shows that separate DNNs were trained to infer QMAI-fibrosis in tissue stained with hematoxylin and eosin (H&E). Models based on GNNs were trained to predict slide-level CRN fibrosis score from iQMAI using, 500 MT-stained whole-slide images (split evenly across CRN 0-4) from two completed NASH clinical trials were divided into training (70%), validation (15%), and test (15%) sets.

Paired QMAI and WSI of H&E- or MT-stained tissue were used to train iQMAI (during training), which are then deployed to generate inferred QMAI-like images generated overlay. (during deployment), in accordance with the models described above in connection with FIGS. 1-1A and 1-1B. GNNs, trained on CNN-generated pixel-level overlays of NASH fibrosis or on inferred QMAI images, predict slide-level CRN fibrosis score. Fibrosis overlays were generated for each slide via ML-QMAI and AIM-NASH. GNNs predicted CRN fibrosis stage based on these overlays. Test set performance was assessed by comparing model CRN fibrosis grade predictions against scores provided by the trials' central pathologist (CP) via kappa statistics (linear Cohen's). In the test set, concordance between CP assessment and ML-QMAI fibrosis stage predictions was moderately high (kappa=0.696). Furthermore, ML-QMAI predictions as concordant with CP as AIM-NASH and CP (kappa=0.695). Utilizing QMAI fibrosis overlays in conjunction with MT-ML-overlays to train the GNNs (not shown in FIG. 1-3) yielded improved concordance with CP scoring (kappa=0.721). FIG. 1-4 illustrates a region of interest highlighting fibrosis in NASH tissue stained with MT (left), (b) as an MLQMAI overlay (center), and (c) as an AIM-NASH generated overlay.

### II. Stromal Subdivisional Model

In some embodiments, method, system and computer readable storage medium are provided that use a machine learning (ML) model to segment a pathology slide image into a plurality of portions each corresponding to one of the plurality of stromal sub-types of cancer stroma. In some examples, the pathology slide image may be any suitable slide image, such as a WSI (e.g., H&E-stained image). The plurality of stromal sub-types may include at least five stromal sub-types based on histological appearance, including densely inflamed stroma, densely fibroblastic stroma, mature stroma, immature stroma, and elastosis. Thus, the ML model may be trained to divide an image region into at least these five sub-types, which results in each of the segmented portions in the pathology slide image corresponding to one of the five stromal sub-types. It is appreciated that the ML model may be trained to divide an image region into fewer or more than five sub-types. The training of the ML model is further described in detail.

In some embodiments, training the ML model for determining the stromal sub-types may use a plurality of annotations made by human experts (e.g., pathologists). An annotation software tool may be provided to these human pathologists to annotate one or more training slide images (e.g., WSI images). The training slide image may be stained (e.g., H&E-stained). Each annotation made by a human pathologist may include an association between an image region (e.g., drawn or labeled by the human pathologist) and one of the sub-types of the cancer stroma based on histological appearance of the image region. In some embodiments, annotations of different sub-types may be obtained from one or more human pathologists annotating on the same training slide image or on different training slide images. For example, a human pathologist may annotate a single training slide image with multiple annotations corresponding to different stromal sub-types. Another human pathologist may annotate the same training slide image also with multiple annotations. As a result, annotations from different human pathologists may be collected and provided to train the ML model.

Alternatively, and/or additionally, a human pathologist may annotate one or more training slide images each with multiple annotations corresponding to a subset of the plurality of stromal sub-types (e.g., one class, two classes, three classes etc.). In other words, a human pathologist may not need to annotate one entire training image exhaustively for all sub-types even if all of the stromal sub-types can be visualized. Allowing the pathologist to annotate a subset of the plurality of sub-types per training slide image enables the pathologist to focus on a few sub-types with sufficient details in the training image and ignore to annotate other sub-types with less details. The collected annotations from multiple human pathologists on multiple training slide images will include annotations for all sub-types of the plurality of stromal sub-types. Such training method helps to obtain more accurate annotations collectively from multiple human pathologists annotating multiple training images than from a single human pathologist and/or annotating a single training slide image.

Although training of a ML model for five stromal sub-types is described, it is appreciated that any suitable ML model can be trained to divide an image region into additional sub-types for cancer stroma, such as six, seven, or more sub-types. Similarly, the ML model may be trained to divide an image region into a subset of the plurality of sub-types (e.g., four or fewer than four sub-types).

In some embodiments, a software may be provided for assisting human pathologists to annotate the training slide images. For example, a human pathologist may be guided to focus on a subset of the plurality of stromal sub-types, for example, certain classes, based on the type of ML model to be trained (e.g., for certain types of tumor), population of patients responding to certain therapies or treatments. In a non-limiting example, a human pathologist may be guided to focus on certain stromal sub-types related to a particular type of tumor.

In some embodiments, a software may also be provided to overlay various segmented portions obtained from the ML model for stromal subdivision onto the original pathology slide image. For example, each of the segmented portions may correspond to one of the plurality of stromal sub-types (e.g., five sub-types) and represented in a respective color. The segmentation of the stromal sub-types may enable certain human interpretable features (HIFs) to be extracted and used to further quantify the stromal sub-types to predict prognosis, gene expression, and/or other clinically relevant features associated with solid tumors, where is further described in the present disclosure.

In some embodiments, the ML model as described for subdivision can be combined with one or more other machine learning models and used to predict prognosis, gene expression, and/or other clinically relevant features associated with solid tumors. For example, the method, system and computer readable storage medium as described herein may additionally provide a second ML model to determine one or more cancer-associated stroma areas in a pathology slide image, and provide the one or more cancer-associated stroma areas as input to the ML model for subdivision of cancer stroma. In some examples, the second ML model may be a tissue-type model, the details of which will be further described herein in the present disclosure. In such configuration, the ML model for stromal subdivision may be trained using cancer-associated stroma areas determined by the second ML model. As a result, the accuracy of the inference from the ML model is improved because the input to the ML model is restricted only to certain tissue types such as cancer-associated stroma.

In some embodiments, the method, system, and computer readable storage medium as described herein may predict prognosis, gene expression, and/or other clinically relevant features associated with solid tumors based on the plurality of segmented regions in the pathology slide image obtained using the ML model for the stromal subdivision as described above and further described herein, where each of the segmented regions corresponds to one of the plurality of stromal sub-types as described herein. In performing prediction, one or more human interpretable features (HIFs) may be determined based at least in part on the plurality of segmented regions to quantify the stromal sub-types. The HIFs may subsequently be used to predict prognosis, gene expression, and/or other clinically relevant features associated with solid tumors. In some embodiments, the HIFs may include the areas of a particular tissue type, and/or a ratio of areas of different tissue types that are shown to correlate to prognosis, gene expression, and/or other clinically relevant features associated with solid tumors.

In some embodiments, the HIFs may include total area, ratio of total area, or area proportion related to one or more stromal sub-types described herein in the present disclosure. In some non-limiting examples, the HIFs may include one or more of total area of a stromal sub-type, area proportion of a stromal sub-type over total tissue, area proportion of a stromal sub-type over total stroma, area proportion of a stromal sub-type over cancer, ratio of total area of a stromal sub-type to another stromal sub-type, and/or total area or area proportion of a combination of two or more stromal sub-types over total tissue, total stroma or cancer. For example, the HIFs may include total area or area proportion related to specific stromal sub-type, such as: total area of mature stroma, area proportion of mature stroma over total tissue, area proportion of mature stroma over total stroma, and/or area proportion of mature stroma over cancer. In other examples, the HIFs may also include area proportion of all possible combinations of the various stromal sub-types described herein in the present disclosure, such as, for example, mature stroma/immature stroma, mature stroma/elastosis, etc. In other examples, the HIFs may also include area proportion of any combination of two or more sub-types of stroma (for example, mature and fibroblastic stroma) over total tissue, total stroma or cancer.

In non-limiting examples, the types of cancer disease that may be predicted using the HIFs may include but not limited to one or more of: non-small cell lung cancer (NSCLC), pancreatic adenocarcinoma, cholangiocarcinoma, colorectal carcinoma, urothelial carcinoma, and breast cancer.

In some embodiments, a statistical model may be trained to correlate certain HIFs with certain types of prognosis, gene expression, and clinically relevant features associated with certain types of solid tumor. For example, Lung adenocarcinoma TCGA samples showed that higher combined proportional areas of mature and fibroblastic stroma relative to total cancer stroma was associated with poor overall survival (OS) (p=0.007), while higher combined proportional areas of densely inflamed stroma and elastosis relative to total cancer stroma was associated with improved OS (p=0.007). Thus, the statistical model may be trained to correlate these HIFs with the overall survival.

Additionally, and/or alternatively, combined area proportion of fibroblastic stroma and mature stroma over cancer stroma, along with area proportion of fibroblastic stroma over total tumor were shown to be positively associated with a pan-cancer proliferative CAF gene expression signature. This signature has been found to be associated with poor prognosis across several cancer types.

In some embodiments, the HIFs may additionally include cellular HIFs to gain additional quantification of the stromal environment. For example, the method, system, and computer readable storage medium as described herein may additionally/alternatively use a third ML model to predict one or more cells in a pathology slide image, and use the one or more predicted cells to determine the cellular HIFs. The third ML model may be a cell-type model, which will be further described in detail. In some embodiments, the tissue-type ML model and the cell-type ML model may be independent ML models. In some embodiments, the tissue-type ML model and the cell-type ML model may be combined in one ML model.

In some embodiments, the cellular HIFs may include one or more of: total count of a cell type in a stromal sub-type, count proportion of a cell type over another cell type in a stromal sub-type, or density of a cell type in a stromal sub-type, and a combination of total count/count proportion/density of cell type(s) in two or more stromal sub-types. For example, some cellular HIFs may include one or more of: total macrophage count in immature stroma, count proportion of macrophages over fibroblasts in immature stroma, or density of macrophages in immature stroma.

Additionally, and/or alternatively, within fibroblastic stroma, count proportion of fibroblast cells over all predicted cells was positively associated with a pan-cancer activated CAF gene expression signature pan-myCAF in TCGA Lung adenocarcinoma samples. On the other end, count proportion of lymphocytes over all predicted cells was significantly negatively associated with pan-myCAF.

Whereas embodiments for predicting prognosis, gene expression, and/or other clinically relevant features associated with solid tumors based on subdivision of cancer stroma are described herein, it is appreciated that variations of these embodiments are also possible. In a non-limiting example, method, system, and computer readable storage medium may be provided to: use a tissue-type ML model to determine one or more cancer-associated stroma areas in a pathology slide image; use a cancer stroma subdivision ML model to segment the pathology slide image into a plurality of portions based at least in part on the one or more cancer-associated stroma areas as input to the second ML model; use a cell-type ML model to predict one or more cells in the pathology slide image; and predict prognosis, gene expression, and/or other clinically relevant features associated with a solid tumor disease based on the plurality of segmented portions and one or more predicted cells in the pathology slide image. As similarly described above, each of the plurality of segmented portions corresponds to one of the plurality of stromal sub-types. In some embodiments, predicting the prognosis, gene expression, and/or other clinically relevant features associated with the solid tumor disease may include: determining one or more human interpretable features (HIFs) based at least in part on the plurality of segmented regions; and predicting the prognosis, gene expression, and/or other clinically relevant features based at least in part on the one or more HIFs. The one or more HIFs may additionally include cellular HIFs based on the predicted cells from the cell-type ML model.

As described above with other embodiments, the plurality of stromal sub-types comprises at least densely inflamed stroma, densely fibroblastic stroma, mature stroma, immature stroma, and elastosis. The solid tumor diseases that the various embodiments as described herein may be applied include, but not limited to, one or more of: NSCLC, pancreatic adenocarcinoma, cholangiocarcinoma, colorectal carcinoma, urothelial carcinoma, and breast cancer.

As described above with other embodiments, the HIFs may include total area, ratio of total area, or area proportion related to one or more stromal sub-types described herein in the present disclosure. In some non-limiting examples, the HIFs may include one or more of total area of a stromal sub-type, area proportion of a stromal sub-type over total tissue, area proportion of a stromal sub-type over total stroma, area proportion of a stromal sub-type over cancer, ratio of total area of a stromal sub-type to another stromal sub-type, and/or total area or area proportion of a combination of two or more stromal sub-types over total tissue, total stroma or cancer. For example, the HIFs may include total area or area proportion related to specific stromal sub-type, such as: total area of mature stroma, area proportion of mature stroma over total tissue, area proportion of mature stroma over total stroma, and/or area proportion of mature stroma over cancer. In other examples, the HIFs may also include area proportion of all possible combinations of the various stromal sub-types described herein in the present disclosure, such as, for example, mature stroma/immature stroma, mature stroma/elastosis, etc. In other examples, the HIFs may also include area proportion of any combination of two or more sub-types of stroma (for example, mature and fibroblastic stroma) over total tissue, total stroma or cancer.

Similar to other embodiments described above, in some embodiments, the one or more cellular HIFs may include one or more of: total count of a cell type in a stromal sub-type, count proportion of a cell type over another cell type in a stromal sub-type, or density of a cell type in a stromal sub-type, and a combination of total count/count proportion/density of cell type(s) in two or more stromal sub-types.

The various embodiments described herein are advantageous over existing systems. For example, the subdivision model captures the diversity of stromal architecture and histological appearance with higher granularities of details, which enables quantification of stromal subtypes via HIFs for improved prediction of prognosis, gene expression, and/or other clinically relevant features associated with solid tumors. Further, the subdivision ML model can be combined with additional ML models to improve the performance of prediction.

Still other aspects, embodiments, and advantages of these exemplary aspects and embodiments, are discussed in detail below. The accompanying drawings are included to provide illustration and a further understanding of the various aspects and embodiments and are incorporated in and constitute a part of this specification. The drawings, together with the remainder of the specification, serve to explain principles and operations of the described and claimed aspects and embodiments.

FIG. 2-1 shows components of a deploying system 200 for prognostic prediction, diagnostic prediction, or patient outcome prediction, gene expression prediction or predicting other clinically related features of solid tumors using at least a stroma subdivision model, in accordance with some embodiments of the technology described herein. The deployment system 200 may be configured to implement any of the methods described in various embodiments in the present disclosure. In some embodiments, the deployment system 200 may include a stroma subdivision model 208 configured to segment a pathology slide image into a plurality of portions each corresponding to a respective stroma sub-type of the plurality of stromal sub-types for cancer stroma. The stroma subdivision model 208 may be a machine learning model described as above. For example, the subdivision model 208 may be a neural network, such as a convolutional neural network (CNN), or other suitable neural networks. In some examples, the pathology slide image may be any suitable slide image such as a WSI (e.g., H&E-stained image). The plurality of stromal sub-types may include at least five stromal sub-types based on histological appearance, including densely inflamed stroma, densely fibroblastic stroma, mature stroma, immature stroma, and elastosis.

In some embodiments, densely inflammatory stroma include stroma showing high density of inflammatory cells, such as lymphocyte, plasma cell, neutrophil or eosinophil rich or a have mixture of two or more cell types. Densely fibroblastic stroma may include stroma showing high density of fibroblast and myofibroblast devoid of mature collagen, which usually shows a fascicular arrangement, and less commonly appears less organized. Mature stroma may include mature collagen fibers with fibrocytes stratified into multiple layers, it also consists of collagen that is broad with hyalinization. The density of fibrocytes is less than that seen in densely fibroblastic. Immature stroma may include randomly oriented fibroblasts/fibrocytes in a myxoid stroma with no mature collagen fibers. Elastosis may include stroma showing accumulation of a large amount of elastin fibers that are secreted by stromal cells like fibroblasts and myofibroblasts. Examples of these stromal sub-types are shown in FIG. 2-2.

With further reference to FIG. 2-1, the subdivision model 208 may be trained using annotations from human pathologists on training pathology slide images as described herein in the present disclosure. Although five sub-types of stromal are described and shown, it is appreciated that different stromal sub-types may be admixed or blended into each other to additional sub-types of stroma. As a result, the subdivision model may be trained to segment a pathology slide image into more than five stromal sub-types.

Additionally, system 200 may include a human interpretable feature (HIF) extractor 210. Feature extractor 210 may extract HIFs (e.g., area proportion, total area, etc.) from the subtypes output by the stroma subdivision model 208 to further quantify the areas of stromal subtypes. For example, as described above, the examples of HIFs may include one or more of total area of mature stroma, area proportion mature stroma over total tissue, area proportion mature stroma over total stroma, or area proportion mature stroma over cancer. These quantified features can be associated with prognosis, gene expression, or other clinically relevant features associated with solid tumors. Thus, the quantified features may be used to predict prognosis, gene expression, or other clinically relevant features associated with cancer. In non-limiting examples, the types of cancer disease that may be predicted using the HIFs may include one or more of: NSCLC, pancreatic adenocarcinoma, cholangiocarcinoma, colorectal carcinoma, urothelial carcinoma, and breast cancer.

With reference to FIG. 2-1, system 200 may include a statistical model 212, which may be trained to correlate certain HIFs with certain types of prognosis, gene expression, and clinically relevant features associated with certain types of solid tumor. For example, the statistical model may be trained to provide result based at least in part on the extracted HIFs. In a non-limiting example, a higher combined proportional areas of mature and fibroblastic stroma relative to total cancer stroma may indicate a poor overall survival for lung adenocarcinoma patients, whereas a higher combined proportional areas of densely inflamed stroma and elastosis relative to total cancer stroma may indicate an improved overall survival.

With reference to FIG. 2-1, system 200 may include one or more additional ML models, which may be combined with the stroma subdivision model 208. In some embodiments, system 200 may include a tissue-type model 202 that is trained to identify multiple tissue regions including cancer, necrosis, and cancer stroma, etc. in a pathology slide image. In some embodiments, system 200 may provide one or more tissue regions corresponding to predicted cancer-associated stroma areas (from tissue-type model 202) as input to the stroma subdivision model 208.

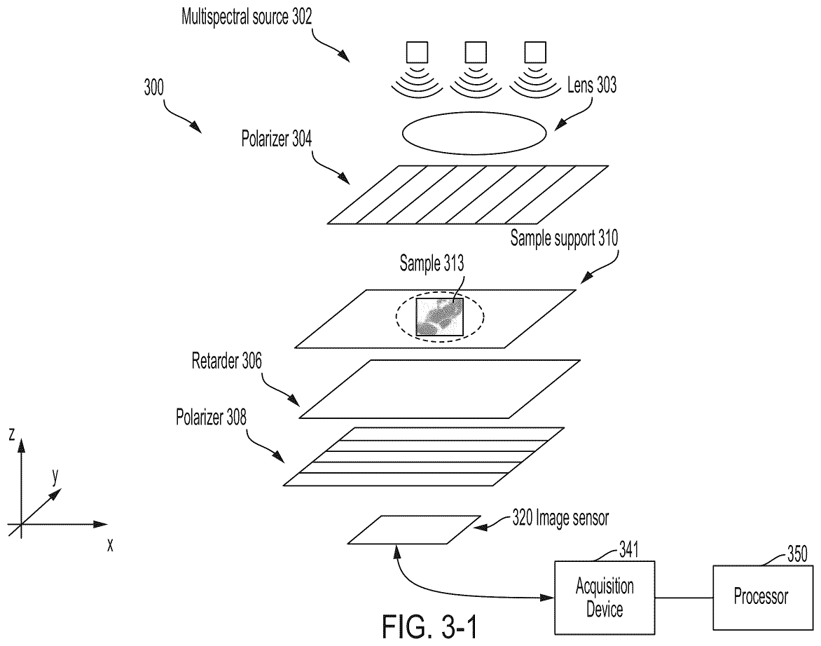

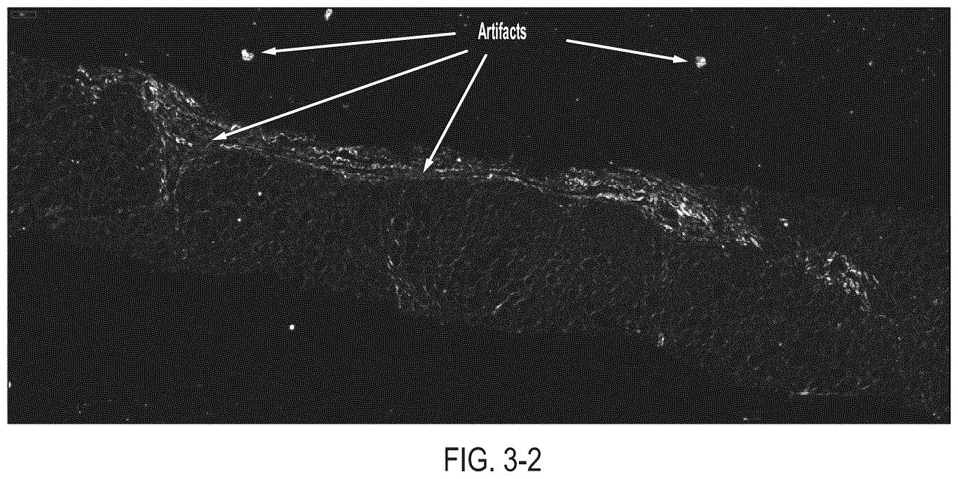

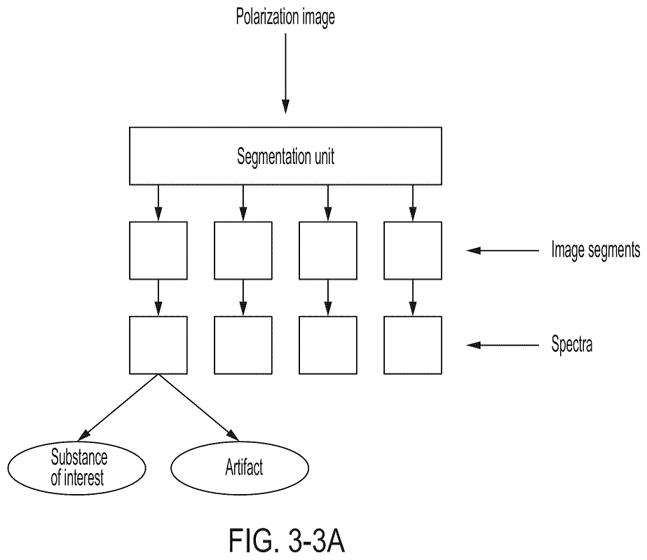

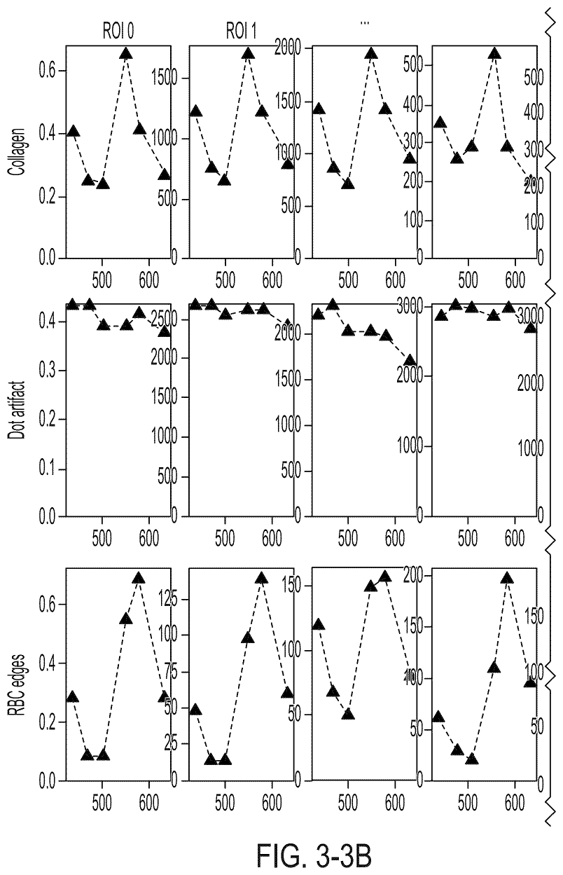

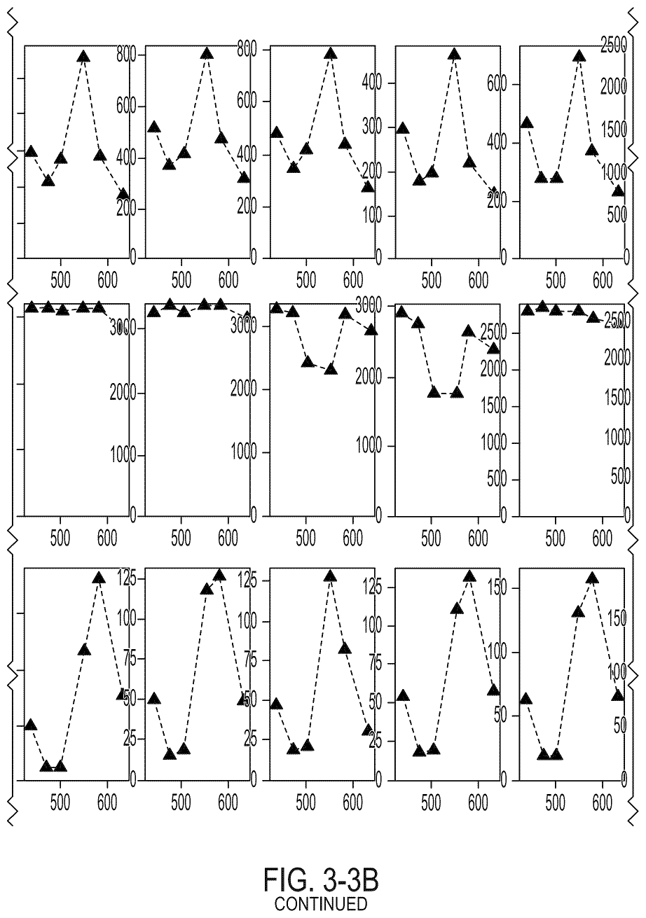

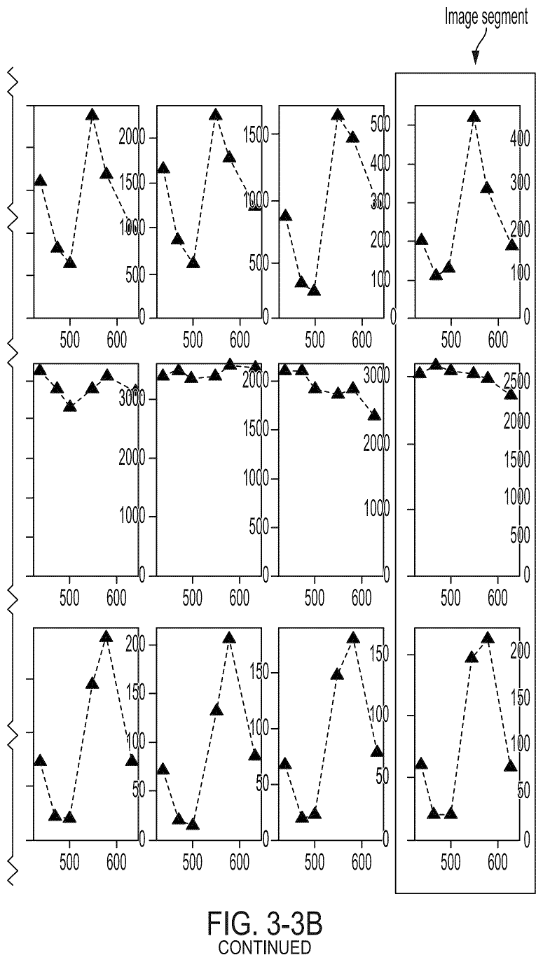

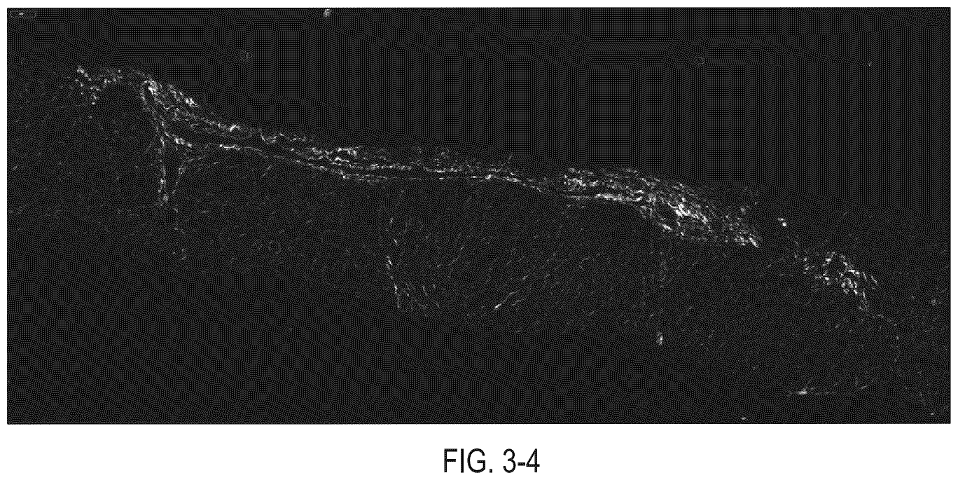

FIG. 2-3A shows an example of PDAC tissues, with segmentation of necrosis, cancer, and cancer stroma. In some examples, the segmentation result in FIG. 2-3A may be obtained using the tissue-type model 202. Then, the subdivision model 208 may subdivide the cancer stroma areas into one of the plurality of stromal sub-types based on the histological appearance of the stroma. FIG. 2-3A is presented in colors. Specifically, necroses are depicted in black, stroma is depicted in orange, cancer is depicted in red, artifacts are depicted in yellow and the background is depicted in grey.

FIG. 2-3B shows an example of PDAC stromal sub-types using the subdivision model 208. FIG. 2-3B is also presented in colors. Elastoses are depicted in blue, fibroblastic stroma is depicted in light green, densely inflamed stroma is depicted in cyan, immature stroma is depicted in dark green and mature stroma is depicted in orange.

Additionally, and/or alternatively, system 200 may include another ML model, e.g., cell-type model 204, which can be trained to predict one or more cells in a pathology slide image. The HIF extractor 210 may additionally use the one or more predicted cells to determine cellular HIFs, the examples of which include one or more of: total count of a cell type in a stromal sub-type, count proportion of a cell type over another cell type in a stromal sub-type, or density of a cell type in a stromal sub-type, and a combination of total count/count proportion/density of cell type(s) in two or more stromal sub-types. For example, some cellular HIFs may include one or more of: total macrophage count in immature stroma, count proportion of macrophages over fibroblasts in immature stroma, or density of macrophages in immature stroma.

Thus, the HIFs output from the HIFs extractor 210 may include both tissue and cellular HIFs based on the sub-type stroma in the pathological slide (e.g., via stroma subdivision model 208) and one or more predicted cells in the pathology slide image (e.g., via cell-type model 204). In some embodiments, the HIFs may include tissue specific features (e.g., total area HIFs based only on stromal subdivision, e.g., output from stromal subdivision model 208). Alternatively, and/or additionally, the HIFs may include tissue specific area proportion HIFs using a combination of stromal subdivision (e.g., output from stromal subdivision model 208) and predicted tissue types (e.g., output from tissue-type model 202). Alternatively, and/or additionally, the HIFs may include cell and tissue combined features based on a combination of cell model count, count proportion, density, ratio HIFs in specific stromal sub-type areas.

These features can be correlated to prognosis, gene expression, and/or other clinically relevant features associated with solid tumors via the statistical model 212 and used for the prediction of thereof. As described above in the present disclosure, the solid tumor disease for which system 200 may be applied include one or more of: NSCLC, pancreatic adenocarcinoma, cholangiocarcinoma, colorectal carcinoma, urothelial carcinoma, and breast cancer.

With further reference to FIG. 2-1, tissue-type model 202 and cell-type model 204 are shown to operate independently and separately. It is appreciated that models 202, 204 may also be implemented in one ML model (e.g., 206).

The method and system described herein may be used for predicting prognosis, gene expression, and/or other clinically relevant features associated with solid tumors. It is further appreciated that the system and method described herein may also be applied to prediction of other types of diseases.

Throughout this disclosure, a neural network is used as an exemplary basis for a statistical model (e.g., a deep learning model) that may be used in accordance with some embodiments. However, it should be appreciated that other types of statistical models may alternatively be used, and embodiments are not limited in this respect. Other types of statistical models that may be used include a support vector machine, a neural network, a regression model, a random forest, a clustering model, a Bayesian network, reinforcement learning, metric learning, a genetic algorithm, or another suitable statistical model. Various computer architectures/systems may be used to implement the deployment system (200 in FIG. 2-1) and training system thereof (not shown), and/or any components thereof.

As an example, the stroma subdivisional models described herein may be used to predict stromal composition and prognosis in non-small cell lung cancer (NSCLC) from hematoxylin and eosin (H&E)-stained tissue. A convolutional neural network-based model was developed to classify CAS as immature, mature, densely inflamed, densely fibroblastic, or elastosis. This model was trained using manual pathologist-derived annotations (N=3019) of H&E-stained whole slide images (WSIs) of PDAC obtained from the TCGA (N=126). This stromal subdivision model was deployed on H&E-stained Potentiated lung adenocarcinoma (LUAD) (N=468) and lung squamous cell carcinoma (LUSC) (N=430) WSIs. Model performance was assessed by qualitative review by expert pathologists. Human interpretable features (HIFs) were extracted from the stromal subdivision model (e.g., proportional area of mature relative to total stroma) and were assessed to identify associations with overall survival (OS) using univariate Cox regression analysis after adjusting for age, sex, and tumor stage.

The stromal subdivision model successfully predicted areas of immature, mature, densely inflammatory, and densely fibrotic stroma, as well as elastosis, in LUAD and LUSC, as shown in FIG. 2-4. FIG. 2-4 illustrates an example of stroma subdivisional model performance in LUAD and LUSC. Overlays show model-identified elastosis, fibroblastic stroma, densely inflamed stroma, immature stroma, and mature stroma (the absence of overlay indicates the presence of normal tissue, cancer or necrosis). As shown in FIG. 2-4, in LUAD, higher combined proportional areas of mature and fibroblastic stroma relative to total cancer stroma was associated with poor OS (p=0.007), while higher combined proportional areas of densely inflamed stroma and elastosis relative to total cancer stroma was associated with improved OS (p=0.007). These findings were validated by stratified tertile analysis based on the corresponding risk direction. Notably, while the average stromal compositions did not differ significantly between NSCLC subtypes, the stromal HIFs were only prognostic in LUAD but not in LUSC. FIG. 2-4 is also presented in colors. As in FIG. 2-3B, elastoses are depicted in blue, fibroblastic stroma is depicted in light green, densely inflamed stroma is depicted in cyan, immature stroma is depicted in dark green and mature stroma is depicted in orange.

In some embodiments, stromal subdivision models may be used in conjunction with an annotation machine learning model that uses multiple imaging modalities, examples of which are described in detail above. The Stromal subdivision model could be used in combination with QMAI or iQMAI models to allow for more detailed evaluation of collagen fibers in stromal histologies not defined entirely by collagen structure. For example, the stromal combined with QMAI or iQMAI can allow for quantification of collagen in densely inflamed or densely fibroblastic areas of stroma.

### III. Quantitative Polarization Imaging

Described herein are techniques for denoising images of samples produced using polarization microscopy. Polarization microscopy is a technique that is particularly suitable to image birefringent samples (samples having a refractive index that depends on the polarization and propagation direction of incident light). Birefringence is responsible for double refraction, the phenomenon by which a ray of light, when incident upon a birefringent material, is split by polarization into two rays taking slightly different paths. When used to image birefringent samples, polarization microscopy enhances contrast to a greater extent than imaging techniques such as darkfield and brightfield illumination, differential interference contrast, phase contrast, Hoffman modulation contrast, and fluorescence.

A simple polarization-sensitive microscope may be formed by placing perpendicularly oriented polarization filters into the beam of light before and after it passes the sample. Without any birefringent samples, light passing the first filter is nearly totally blocked by the second filter. When birefringent samples are placed in the illumination path, the polarization state of light that passes through them changes, allowing some component of that light to pass the second filter.

Collagen is known to exhibit birefringence. Collagen is a structural protein that is found in various connective tissues. Collagen is made of amino acids connected together to form a triple helix of elongated fibril, called collagen helix. Collagen is found in cartilage, bones, tendons, ligaments, skin, corneas, blood vessels, the gut, intervertebral discs, and the dentin in teeth. Studies have linked defects of collagen to the development of diseases. Thus, detection of collagen is often used in medical diagnosis. Amyloids also exhibit birefringence. Amyloids are particular aggregates of proteins characterized by their ability to be stained by certain dyes. Studies have linked amyloids to the development of some neurodegenerative diseases. The main advantage of polarization microscopy over other types of imaging techniques is that it allows for the characterization of biological samples without having to stain them.

Multispectral polarization microscopy involves illumination of a sample using polychromatic light. This can be achieved using banks of light emitting diodes (LEDs) or other optical sources emitting at different wavelengths or a broadband light source with a plurality of narrowband color filters. Because different substances generally exhibit different spectral profiles, multispectral polarization microscopy allows for the characterization of different substances.

The inventors have recognized and appreciated that raw polarization images captured by polarization microscopy contain not only signals produced from polarization, but also signals produced from scattering by artifacts (e.g., crystals and/or hemosiderin). These off-target signals reduce the signal-to-noise ratio (SNR) of the birefringent substance of interest, and reduce the utility and accuracy of polarization microscopy for substance quantification. Although various techniques have been developed for quantitative polarization imaging, these techniques have not addressed SNR reduction due to scattering and have been deployed primarily for unstained specimens.

The techniques developed by the inventors and described herein reduce noise resulting from scattering, thus enabling much cleaner, noise-reduced images. The images so produced can be either directly visualized by a medical practitioner or used for downstream machine-learning models. Moreover, the techniques described herein enable segmentation of different substances with minimal or no annotation, and may be used to image collagen, amyloids or other birefringent specimens. In some embodiments, noise reduction involves i) dividing an image in segments (e.g., pixels or groups of pixels), ii) performing spectral analysis of each segment, and iii) separating each segment on the basis of its spectral profiles. In this way, it can be determined in which segments artifacts are present. Segments in which artifacts are present can be discarded or altered. The process by which each segment is separated on the basis of its spectral profile can be performed in various ways. In some embodiments, the spectrum associated with a segment can be expressed as the weighted combination of the spectra of known substances. For example, the spectrum associated with a segment can be expressed as the weighted combination of the spectrum of collagen (the substance of interest) with the spectrum of calcium (the expected scatterer). Optionally, if the types of scatterers are not known a priori, a machine learning algorithm may be trained to identify which substances (based on their spectra) are present in an image segment. In other words, instead of manually annotating the substances, a machine learning algorithm is trained to perform annotation in an automatic fashion.

In an aspect of the present disclosure, a method is provided that performs multi-spectral polarization imaging of a sample to generate a polarization image of the sample; segments the polarization image to form a plurality of image segments; obtains spectral characteristics associated with at least some of the plurality of image segments, wherein obtaining the spectral characteristics comprises performing spectral analysis on the at least some of the plurality of image segments; and identifies, using the respective spectral characteristics, a first subset of the at least some of the plurality of image segments as including a substance of interest and a second subset of the at least some of the plurality of image segments as including artifacts.

In some embodiments, segmenting the polarization image to form the plurality of image segments comprises segmenting the polarization image pixel-wise so that each image segment corresponds to a pixel of the polarization image. Alternatively, segmenting the polarization image to form the plurality of image segments comprises segmenting the polarization image pixel-wise so that each image segment corresponds to a group of pixels of the polarization image. As further discussed below, spectral analysis is ultimately performed on each image segment to identify what substance(s) are present in that segment.

In some embodiments, the method further comprises generating a denoised image of the sample using the first subset (and discarding the second subset). Optionally, the denoised image of the sample may be provided as input to a machine learning model. Alternatively, the denoised of the sample may be used for visual inspection by a medical practitioner.

It should be noted that the denoising techniques described herein may be applied to imaging systems other than polarization microscopy. For example, these techniques may be used in conjunction with phase-based imaging, fluorescence, autofluorescence, second harmonic generation, and/or brightfield imaging. Information obtained using any one of the approaches may be with multispectral imaging to generate a hypercube of aligned data for rich substance clustering.

Still other aspects, embodiments, and advantages of these exemplary aspects and embodiments, are discussed in detail below. The accompanying drawings are included to provide illustration and a further understanding of the various aspects and embodiments and are incorporated in and constitute a part of this specification. The drawings, together with the remainder of the specification, serve to explain principles and operations of the described and claimed aspects and embodiments.

FIG. 3-1 illustrates a system for multispectral polarization microscopy, in accordance with some embodiments of the technology described herein. System 300 is configured to image sample 313, which may include a birefringent substance (e.g., collagen or amyloids). System 300 includes multispectral source 302, lens 303, input polarizer 304, sample support 310 on which sample 313 is disposed, retarder 306, output polarizer 308, image sensor 320, acquisition device 341 and processor 350. Sample support 310 may be rotatable with respect to the z-axis to facilitate orientation of the sample. Input polarizer 304 may be a broadband polarizer. Although not illustrated, a lens (e.g., objective lens or imaging lens) may be present between sample support 310 and image sensor 320.

Multispectral source 302 may be configured to emit polychromatic light. The spectrum of emission of multispectral source 302 may be continuous, or may consist of discrete bands. The spectrum may span part of (or the entirety of) the visible range. Optionally, the spectrum may extend into part of the ultraviolet band and/or the infrared band. In some embodiments, multispectral source 302 includes multiple LEDs emitting at mutually distinct wavelengths.

Input polarizer 304 works in combination with output polarizer 308 to block light emitted directly by multispectral source 302 and to permit passage of light produced as a result of the birefringence of sample 313. In some embodiments, input polarizer 304 and output polarizer 308 exhibit polarization axes that are orthogonal to one another (though not all embodiments are limited in this respect). For example, in the implementation of FIG. 3-1, the polarization axis of input polarizer 304 is oriented parallel to the y-axis and the polarization axis of output polarizer 308 is oriented parallel to the x-axis. Optionally, a retarder 306 is placed between the polarizers, to enhance optical path differences in the sample. The retarder may be an anisotropic plate that presents different refractive indices along different directions.

When illuminated with polarized light, sample 313 produces two wave components polarized in mutually orthogonal planes. Due to the birefringent nature of sample 313, the optical path length of these components may be different and may vary with the propagation direction through the sample. After exiting the sample, the light components may be out of phase relative to one another. As the output light passes through output polarizer 308, those components recombine through constructive and destructive interference. The maximum degree of brightness can be achieved when sample 313 is oriented at a 45-degree angle with respect to the polarization axes of the two polarizers.

Image sensor 320 may detect the image produced by sample 313. Image sensor 320 may be implemented in various ways, including for example a complementary metal-oxide-semiconductor (CMOS) device or a charged-coupled device (CCD). Acquisition device 341 may form a digital image and processor 350 may be configured to perform denoising, as described in detail further below.

FIG. 3-2 illustrates an image of a sample obtained using polarization microscopy, in accordance with some embodiments of the technology described herein. FIG. 3-2 is an image of human tissues. Contrast in the image is due in part to the birefringent nature of the sample. In addition to imaging the areas of interest (e.g., collagen), unwanted artifacts also appear in the image. These artifacts may be due to a variety of reasons, including for example due to optical scattering. When illuminated, certain particles (e.g., calcium) scatter the input light, leading to the artifacts shown in FIG. 3-2. The presence of artifacts reduces the overall SNR of the image. This can negatively affect the ability of a medical practitioner to perform medical diagnosis on the image. Further, if the image is intended to be provided as input to a machine learning model, the low SNR can negatively affect the performance of the model.

The inventors have developed methods for denoising polarization images. FIG. 3-3A is a block diagram illustrating one representative method, in accordance with some embodiments. A polarization image (e.g., obtained using the system of FIG. 3-1) may be provided as input to a segmentation unit. The segmentation unit is configured to segment the polarization image to form a plurality of image segments. The segmentation may be performed pixel-wise, so that each image segment corresponds to a pixel of the polarization image or a group of pixels of the polarization image. Each group may include N×M pixels, although not all groups are limited to rectangular blocks of pixels and different groups of an image may have different dimensions and/or shapes.

Then, spectral analysis is performed on each image segment to identify what substance(s) are present in that segment. Spectra may be obtained by plotting the magnitude of a segment as a function of wavelength, which is enabled by the multispectral nature of the source. Spectral analysis may be performed in various ways. In one example, this involves manual annotation. In some embodiments, the spectrum associated with a segment can be expressed as the weighted combination of the spectra of known substances. For example, the spectrum associated with a segment can be expressed as the weighted combination of the spectrum of collagen (the substance of interest) with the spectrum of calcium (the expected scatterer). By comparing the spectrum associated with a segment with known spectra, the method may determine which substances are present in the segment.

Alternatively, a machine learning algorithm may be trained to identify which substances are present in an image segment. In other words, instead of manually annotating the substances, a machine learning algorithm is trained to perform annotation in an automatic fashion. This may be particularly useful where the types of scatterers are not known a priori.

Based on the spectral characteristics of the image segments, the method identifies a first subset of the image segments as including a substance of interest and a second subset of at least some of the plurality of image segments as including artifacts. A denoised image of the sample may be generated using the first subset (and discarding the second subset). The denoised image may be provided as input to a machine learning model.

FIG. 3-3B illustrates spectra associated with multiple image segments of an image obtained using a polarization imaging apparatus, in accordance with some embodiments of the technology described herein. For each image segment, FIG. 3-3B represents multiple spectra: one spectrum can be traced back to collagen, one spectrum can be traced back to a dot artifact (e.g., calcium) and one spectrum can be traced back to Red blood cells (RBC) edges. Based on these spectra, the method may determine which segments present artifacts, and those segments may be discarded. FIG. 3-4 illustrates an image of a sample obtained using polarization microscopy and upon application of denoising, in accordance with some embodiments of the technology described herein. In particular, FIG. 3-4 represents the image of FIG. 3-2 after processing using the method of FIG. 3-3A. As can be appreciated by comparing FIG. 3-2 with FIG. 3-4, the denoising processing results in the removal of the artifacts, thus improving the overall SNR of the image.

### IV. Overview of Various Concepts

A1. A method comprising: using a machine learning (ML) model to obtain annotations of a pathology slide image obtained in a first imaging modality; wherein the ML model is trained based in part on images obtained from a second imaging modality different from the first imaging modality.

A2. The method of concept A1, wherein the first imaging modality is configured to image a slide based on light source of visible wavelengths and absorption of light by tissue.

A3. The method of concept A2, wherein the second imaging modality comprises one or more of multispectral imaging (MSI), polarization imaging, quantitative phase imaging, or a combination thereof.

A4. The method of concept A3, further comprising training the ML model, using a plurality of pairs of first image and second images; wherein the first image in the pair is obtained from the first modality imaging of a first pathology slide; and the second image in the pair is generated based on a second modality imaging of a second pathology slide corresponding to the first pathology slide.

A5. The method of concept A4, wherein the second pathology slide and the first pathology slide are a same physical slide.

A6. The method of any of concepts A4-A5, wherein the training further includes registering the first image and the second image in each of the pairs of first image and second image.

A7. The method of concept A6, wherein the registering includes aligning the first image and the second image in each of the pairs.

A8. The method of any of concepts A6-A7, wherein the second image in the pair is an annotation image comprising a plurality of objects each associated with a respective portion of the second image.

A9. The method of concept A8, further comprising generating the annotation image by processing an image captured by the second modality imaging over a physical slide.

A10. The method of any of concepts A8-A9, further comprising generating the annotation image based on a plurality of images captured by the second modality imaging over a physical slide.

A11. The method of any of concepts A1-A10, further comprising generating HIFs from the annotations.

A12. The method of concept A11, further comprising using a second ML to predict cell/tissue from the pathology slide image; and generating the HIFs based additionally on the predicted cell/tissue.

A13. The method of any of concepts A11-A12, further comprising predicting a disease based on the HIFs, using a statistical model.

A14. The method of any of concepts A1-A13, wherein the annotations of the pathology slide image comprise heatmaps or labels of tissues/cells in the pathology slide image.

B1. A method comprising using a machine learning (ML) model to obtain annotations of a pathology slide image of a first type; wherein the ML model is trained based in part on training pathology slide images of a second type different from the first type.

B2. The method of concept B 1, wherein the first type of image is obtained from a stained slide; and the second type of image is a stain-invariant image obtained from a triplex slide.

B3. The method of concept B2, wherein the second type of image is a phase image.

C1. A method for denoising images of samples, comprising performing multi-spectral polarization imaging of a sample to generate a polarization image of the sample; segmenting the polarization image to form a plurality of image segments; obtaining spectral characteristics associated with at least some of the plurality of image segments, wherein obtaining the spectral characteristics comprises performing spectral analysis on the at least some of the plurality of image segments; and identifying, using the respective spectral characteristics, a first subset of the at least some of the plurality of image segments as including a substance of interest and a second subset of the at least some of the plurality of image segments as including artifacts.

C2. The method of concept C1, wherein the substance of interest comprises collagen.

C3. The method of any of concepts C1-C2, wherein the substance of interest comprises an amyloid.

C4. The method of any of concepts C1-C3, wherein the artifact comprises calcium.

C5. The method of any of concepts C1-C4, wherein the artifact comprises metal.

C6. The method of any of concepts C1-05, wherein performing multi-spectral polarization imaging of the sample comprises illuminating the sample with a plurality of light emitting diodes (LEDs) emitting light at mutually distinct wavelength simultaneously.

C7. The method of any of concepts C1-C6, wherein performing multi-spectral polarization imaging of the sample comprises illuminating the sample with a plurality of light emitting diodes (LEDs) emitting light at mutually distinct wavelength sequentially.

C8. The method of any of concepts C1-C7, wherein segmenting the polarization image to form the plurality of image segments comprises segmenting the polarization image pixel-wise so that each image segment corresponds to a pixel of the polarization image.

C9. The method of any of concepts C1-C8, wherein segmenting the polarization image to form the plurality of image segments comprises segmenting the polarization image pixel-wise so each image segment corresponds to a group of pixels of the polarization image.

C10. The method of any of concepts C1-C9, further comprising generating a denoised image of the sample using the first subset.

C11. The method of concept C10, further comprising providing the denoised image of the sample as input to a machine learning model.

C12. The method of any of concepts C1-C11, wherein performing spectral analysis on the at least some of the plurality of image segments comprises obtaining spectra associated with the at least some of the plurality of image segments and comparing the spectra to known spectra associated with a plurality of known samples.

D1. A system for denoising images of samples, comprising a multi-spectral polarization imaging apparatus configured to generate a polarization image of a sample; and a computer hardware processor configured to segment the polarization image to form a plurality of image segments; obtain spectral characteristics associated with at least some of the plurality of image segments, wherein obtaining the spectral characteristics comprises performing spectral analysis on the at least some of the plurality of image segments; and identify, using the respective spectral characteristics, a first subset of the at least some of the plurality of image segments as including a substance of interest and a second subset of the at least some of the plurality of image segments as including artifacts.

D2. The system of concept D1, wherein the substance of interest comprises collagen.

D3. The system of any of concepts D1-D2, wherein the substance of interest comprises an amyloid.

D4. The system of any of concepts D1-D3, wherein the artifact comprises calcium.

D5. The system of any of concepts D1-D4, wherein the artifact comprises metal.

D6. The system of any of concepts D1-D5, wherein the multi-spectral polarization imaging apparatus comprises a plurality of light emitting diodes (LEDs) emitting light at mutually distinct wavelength simultaneously, and wherein the system further comprises a controller configured to cause the LEDs to emit light simultaneously.

D7. The system of any of concepts D1-D6, wherein the multi-spectral polarization imaging apparatus comprises a broadband light source, and a plurality of narrowband color filters.

D8. The system of any of concepts D1-D7, wherein the multi-spectral polarization imaging apparatus comprises a plurality of light emitting diodes (LEDs) emitting light at mutually distinct wavelength simultaneously, and wherein the system further comprises a controller configured to cause the LEDs to emit light in accordance with time-domain multiplexing (TDM).

D9. The system of any of concepts D1-D8, wherein segmenting the polarization image to form the plurality of image segments comprises segmenting the polarization image pixel-wise so that each image segment corresponds to a pixel of the polarization image.

D10. The system of any of concepts D1-D9, wherein segmenting the polarization image to form the plurality of image segments comprises segmenting the polarization image pixel-wise so each image segment corresponds to a group of pixels of the polarization image.

D11. The system of any of concepts D1-D10, wherein the processor is further configured to generate a denoised image of the sample using the first subset.

D12. The system of any of concepts D1-D11, wherein performing spectral analysis on the at least some of the plurality of image segments comprises obtaining spectra associated with the at least some of the plurality of image segments and comparing the spectra to known spectra associated with a plurality of known samples.

E1. A method comprising using a machine learning (ML) model to segment a pathology slide image into a plurality of portions, wherein the ML model is configured to divide an image region into a plurality of regions corresponding to a plurality of stromal sub-types comprising at least densely inflamed stroma, densely fibroblastic stroma, mature stroma, immature stroma, and elastosis; and each of the plurality of segmented portions corresponds to one of the plurality of stromal sub-types.

E2. The method of concept E1, wherein the ML model is a first ML model, and wherein the method further comprises using a second ML model to determine one or more cancer-associated stroma areas in the pathology slide image; and providing the one or more cancer-associated stroma areas as input to the first ML model.

E3. The method of any of concepts E1-E2, further comprising determining one or more human interpretable features (HIFs) based at least in part on the plurality of segmented regions; and predicting prognosis, gene expression, and/or other clinically relevant features based at least in part on the one or more HIFs.

E4. The method of concept E3, wherein the prognosis, gene expression, and/or other clinically relevant features each is associated with one or more of: NSCLC, pancreatic adenocarcinoma, cholangiocarcinoma, colorectal carcinoma, urothelial carcinoma, and breast cancer.

E5. The method of any of concepts E3-E4, wherein the one or more HIFs include one or more of: total area of a stromal sub-type, area proportion of a stromal sub-type over total tissue, area proportion of a stromal sub-type over total stroma, area proportion of a stromal sub-type over cancer, ratio of total area of a stromal sub-type to another stromal sub-type, and/or total area or area proportion of a combination of two or more stromal sub-types over total tissue, total stroma or cancer.

E6. The method of any of concepts E3-E5, wherein the ML model is a first ML model, and wherein the method further comprises: using a second ML model to predict one or more cells in the pathology slide image; and determining the one or more human interpretable features (HIFs) based additionally on the one or more predicted cells.

E7. The method of concept E6, wherein the one or more HIFs additionally include cellular HIFs comprising one or more of: total count of a cell type in a stromal sub-type, count proportion of a cell type over another cell type in a stromal sub-type, or density of a cell type in a stromal sub-type, and a combination of total count/count proportion/density of cell type(s) in two or more stromal sub-types.

E8. The method of any of concepts E1-E7, wherein the plurality of stromal sub-types comprise one or more additional sub-types.

E9. The method of any of concepts E1-E8, wherein the pathology slide image is a H&E-stained image.

F1. A method comprising using a first ML model to determine one or more cancer-associated stroma areas in a pathology slide image; using a second ML model to segment the pathology slide image into a plurality of portions based at least in part on the one or more cancer-associated stroma areas as input to the second ML model, wherein: the second ML model is configured to divide an image region into a plurality of regions corresponding to a plurality of stromal sub-types; and each of the plurality of segmented portions corresponds to one of the plurality of stromal sub-types; using a third ML model to predict one or more cells in the pathology slide image; and predicting prognosis, gene expression, and/or other clinically relevant features associated with a solid tumor disease based on the plurality of segmented portions and the predicted one or more cells in the pathology slide image.

F2. The method of concept F1, wherein the plurality of stromal sub-types comprise at least densely inflamed stroma, densely fibroblastic stroma, mature stroma, immature stroma, and elastosis.

F3. The method of any of concepts F1-F2, wherein predicting the prognosis, gene expression, and/or other clinically relevant features associated with the solid tumor disease based at least in part on the plurality of segmented portions in the pathology slide image comprises determining one or more human interpretable features (HIFs) based at least in part on the plurality of segmented regions; and predicting the prognosis, gene expression, and/or other clinically relevant features based at least in part on the one or more HIFs.

F4. The method of concept F3, wherein the solid tumor disease comprises one or more of: NSCLC, pancreatic adenocarcinoma, cholangiocarcinoma, colorectal carcinoma, urothelial carcinoma, and breast cancer.

F5. The method of any of concepts F3-F4, wherein the one or more HIFs include one or more of: total area of a stromal sub-type, area proportion of a stromal sub-type over total tissue, area proportion of a stromal sub-type over total stroma, area proportion of a stromal sub-type over cancer, ratio of total area of a stromal sub-type to another stromal sub-type, and/or total area or area proportion of a combination of two or more stromal sub-types over total tissue, total stroma or cancer.

F6. The method of concept F5, wherein the one or more HIFs additionally include cellular HIFs comprising one or more of: total count of a cell type in a stromal sub-type, count proportion of a cell type over another cell type in a stromal sub-type, or density of a cell type in a stromal sub-type, and a combination of total count/count proportion/density of cell type(s) in two or more stromal sub-types.

F7. The method of any of concepts F1-F6, wherein the pathology slide image is a H&E-stained image.

### V. Additional Remarks

It is to be appreciated that embodiments of the methods and apparatuses discussed herein are not limited in application to the details of construction and the arrangement of components set forth in the present disclosure or illustrated in the accompanying drawings. The methods and apparatuses are capable of implementation in other embodiments and of being practiced or of being carried out in various ways. Examples of specific implementations are provided herein for illustrative purposes only and are not intended to be limiting. In particular, any embodiment disclosed herein may be combined with any other embodiment in any manner consistent with at least one of the objects, aims, and needs disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described in connection with the embodiment may be included in at least one embodiment. The appearances of such terms herein are not necessarily all referring to the same embodiment.

Also, the phraseology and terminology used herein is for the purpose of description and should not be regarded as limiting. Any references to embodiments or elements or acts of the systems and methods herein referred to in the singular may also embrace embodiments including a plurality of these elements, and any references in plural to any embodiment or element or act herein may also embrace embodiments including only a single element. References in the singular or plural form are not intended to limit the presently disclosed systems or methods, their components, acts, or elements.

Also, various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, or ordinary meanings of the defined terms.

The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present systems and methods or their components to any one positional or spatial orientation.

As referred to herein, the term “in response to” may refer to initiated as a result of or caused by. In a first example, a first action being performed in response to a second action may include interstitial steps between the first action and the second action. In a second example, a first action being performed in response to a second action may not include interstitial steps between the first action and the second action.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

Having thus described several aspects of at least one embodiment, it is to be appreciated that various alterations, modifications, and improvements will readily occur to those skilled in the art. Such alterations, modifications, and improvements are intended to be part of this disclosure and are intended to be within the spirit and scope of the systems and methods described herein. Accordingly, the foregoing description and drawings are by way of example only.

1. A method comprising:
using a machine learning (ML) model to obtain annotations of a pathology slide image obtained in a first imaging modality;
wherein the ML model is trained based in part on images obtained from a second imaging modality different from the first imaging modality.

2. The method of claim 1, wherein:
the first imaging modality is configured to image a slide based on light source of visible wavelengths and absorption of light by tissue.

3. The method of claim 2, wherein:
the second imaging modality comprises one or more of multispectral imaging (MSI), polarization imaging, quantitative phase imaging, or a combination thereof.

4. The method of claim 3, further comprising:
training the ML model, using a plurality of pairs of first image and second images;
wherein:
the first image in the pair is obtained from the first modality imaging of a first pathology slide; and
the second image in the pair is generated based on a second modality imaging of a second pathology slide corresponding to the first pathology slide.

5. The method of claim 4, wherein the second pathology slide and the first pathology slide are a same physical slide.

6. The method of claim 4, wherein:
the training further includes registering the first image and the second image in each of the pairs of first image and second image.

7. The method of claim 6, wherein the registering includes aligning the first image and the second image in each of the pairs.

8. The method of claim 6, wherein the second image in the pair is an annotation image comprising a plurality of objects each associated with a respective portion of the second image.

9. The method of claim 8, further comprising generating the annotation image by processing an image captured by the second modality imaging over a physical slide.

10. The method of claim 8, further comprising generating the annotation image based on a plurality of images captured by the second modality imaging over a physical slide.

11. The method of claim 1, further comprising generating HIFs from the annotations.

12. The method of claim 11, further comprising:
using a second ML to predict cell/tissue from the pathology slide image; and
generating the HIFs based additionally on the predicted cell/tissue.

13. The method of claim 11, further comprising predicting a disease based on the HIFs, using a statistical model.

14. The method of claim 1, wherein the annotations of the pathology slide image comprise heatmaps or labels of tissues/cells in the pathology slide image.

15. A method comprising:
using a machine learning (ML) model to obtain annotations of a pathology slide image of a first type;
wherein the ML model is trained based in part on training pathology slide images of a second type different from the first type.

16. The method of claim 15, wherein:
the first type of image is obtained from a stained slide; and
the second type of image is a stain-invariant image obtained from a triplex slide.

17. The method of claim 16, wherein the second type of image is a phase image.
